(19) United States
(12) Patent Application Publication
(10) Pub. No.: US 2007/0293792 A1
(43) Pub. Date: Dec. 20, 2007

(54) Prostate BPH and tumor detector also useable on other tissues

(76) Inventors: John W. Sliwa, Los Altos, CA (US); Bryan T. Oronsky, Los Altos, CA (US)

Correspondence Address: David W. Collins;Intellectual Property Law, Suite 100, 512 E. Whitehouse Canyon Road, Green Valley, AZ, 85614, US

(21) Appl. No.: 11/818,685

(22) Filed: Jun. 15, 2007

Related U.S. Application Data
(60) Provisional application No. 60/814,626, filed on Jun. 15, 2006.
(60) Provisional application No. 60/857,891, filed on Nov. 8, 2006.

(51) Int. Cl. A61B 5/103

(52) U.S. Cl. 600/587

(57) ABSTRACT

Prostate probe systems are disclosed for assessing one or both of BPH or prostate cancer. The prostate probe systems comprise either a force or pressure sensor mounted on or in a rectally insertable probe or a temperature sensor mounted on or in a rectally insertable probe, or both. Also disclosed are probe systems for evaluating a condition of a prostate gland. Finally, force or hardness mapping devices are disclosed for palpation-examination of patient anatomical tissues for abnormalities or assessing states of firmness.

### CROSS-REFERENCE TO RELATED APPLICATIONS

The present application claims priority from provisional application Ser. No. 60/814,626, filed Jun. 15, 2006, and from provisional application Ser. No. 60/857,891, filed Nov. 8, 2006, the contents of both of which are incorporated herein by reference in their entireties.

### BACKGROUND OF THE INVENTION

The prostate gland in men has had at least two historic issues of relevance to this invention. The first is prostate-cancer wherein one or more tumorous masses or networks of masses develop in the prostate gland. The second is BPH or benign prostatic hyperplasia, which is an age-related enlargement of the prostate organ. Currently, the primary screening technique for these conditions is the digital rectal exam. In this exam, the doctor or clinician places a gloved lubricated finger through the anal sphincter into the rectum and digitally finger-palpates the roof of the rectum to assess both BPH enlargement and the presence of any tumor-like masses. The doctor makes a subjective judgment on both questions based on his/her experience. In general, BPH constitutes an enlargement of the prostate, including downward growth into the rectum and this downward growth is felt as a rectal-wall bulge. Tumors, on the other hand, are typically felt as harder nodules or masses within the lateral confines of the lower surface of the prostate gland also felt directly adjacent the upper rectal wall. The main issue with this current state of affairs is the subjectiveness of it all, often resulting in scatter in the measurements for a given clinician and among different clinicians. Our invention herein removes the subjectiveness from the exams. The invention is also useable on other organs having enlargement or tumor-like mass conditions.

### BRIEF DESCRIPTION OF THE DRAWINGS

We utilize five Figures in explaining the invention as follows:

FIG. 1 depicts a rod-like electronic palpation and/or temperature mapping instrument which is insertable into the rectum for the purpose of one or both of palpating the prostate and/or temperature-mapping the prostate, in accordance with an embodiment of the invention.

FIG. 2 depicts a device similar to that of FIG. 1 inserted in the rectal cavity and palpating and/or temperature mapping the prostate gland through the upper rectal wall.

FIG. 3A depicts force or pressure maps sampled along a detector-row of a force sensor of the palpation device.

FIG. 3B depicts temperature maps sampled along a detector-row of a temperature sensor of the inventive device.

FIG. 4 depicts a finger-mounted palpation and/or temperature mapping device which still involves digital insertion in the rectum but is quantitative, in accordance with another embodiment of the invention.

FIG. 5 depicts a flexible circuit based finger-mounted device before rectal insertion, in accordance with yet another embodiment of the invention.

### DETAILED DESCRIPTION OF THE PREFERRED EMBODIMENTS

We eliminate the subjectiveness from the exam by utilizing a force or pressure sensor which is capable of quantifying the forces or pressures the finger felt in prior art exams. Preferably, the sensor is more sensitive that the manual finger and is provided in the form of an area-wise array of sub-sensors. Before we go any further, when we say pressure or force, unless otherwise stated, we are referring to the mechanical load applied to a given area element. Typically, herein, the area element is a detector sub-element of our detection array. Since the area of those elements is typically fixed, then the force or load on a given element divided by that element's area equals that element's contact pressure.

Our force or pressure sensing array will typically have a large number of sub-elements such as, for example, a 16 by 16 array of sub-elements. Our device will be able to detect the force or pressure on each such sub-element and can form a force or pressure map across the entire array or any portion thereof. The reader will note that the force sensing array is an electronic means of physical palpation which is quantifiable. A preferred force-sensor array vendor is Pressure Profile Systems Inc. (Los Angeles, Calif. 90045). The work done herein was performed using the T2000 and T2500 systems and various TactArray™ sensors having 2 mm pixel pitch and a mini-mum of 256 total pixels of sub-sensors.

Unlike prior art digital finger-palpation, we also include in our prostate exam process a thermographic or temperature mapping capability which detects the hot-spots associated with tumors, infection or disease. This thermographic capability may be used in addition to the force map or separately from the force map. Therefore, the force mapping and the temperature mapping may be done by one device having both detector types or done by separate devices each with only one sensor type. A common handle may also have two attachable sensor types being or may have the two sensors mounted on opposite faces of a single inventive device simultaneously or sequentially. We claim each type of device, pressure-mapping and temperature mapping, separately as well as when combined in one device.

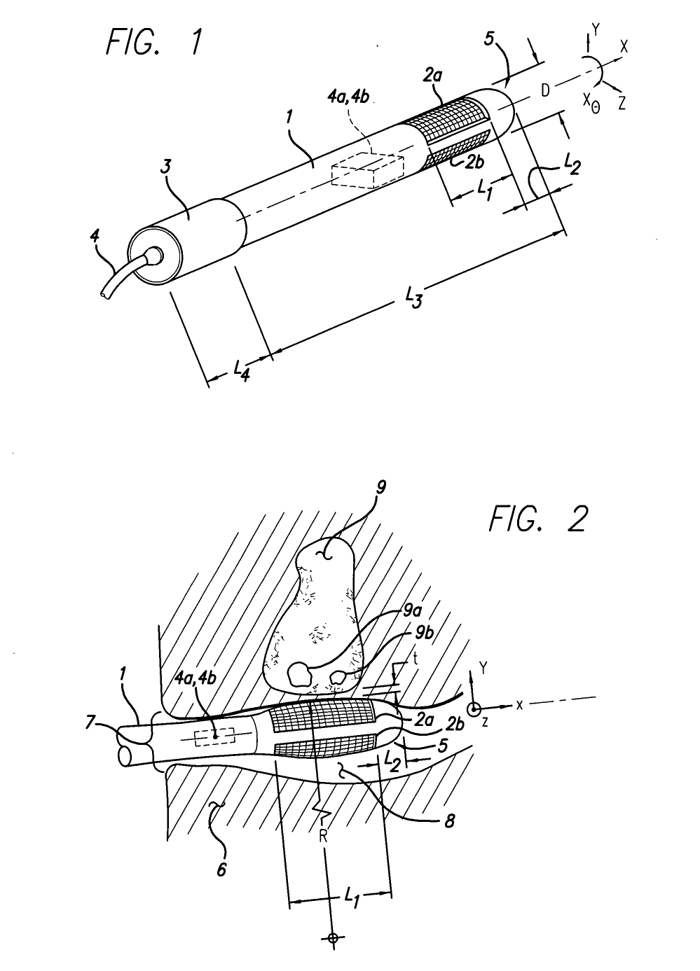

Let us now proceed to FIG. 1. Therein we see an inventive rectal probe 1 for performing both of our force/pressure mapping and our temperature mapping. Probe 1 looks somewhat like an ultrasonic-imaging rectal probe. The probe is depicted as rod-like with an approximate diameter D, an insertable length L3 and a handle length L4. On the insertable length portion L3 we see our two sensors, force/pressure sensor 2a on top and temperature sensor 2b on the bottom. The sensors are shown as having lengths L1 and being oppositely mounted to the cylindrically curved depicted device 1 surface. It will be noted that the sensors are set-back from the probe tip 5 by a distance L2. The probe handle 3 is depicted as having an attached cable or lumen 4 providing power or other support services such as a fluid for lubrication or balloon (not shown) inflation. Note that the probe tip 5 is shown as being approximately hemi-spherically radiused and blended to diameter D for easier rectal insertion. Note also that we depict a probe-related coordinate system with +X being along the insertion direction.

Before going further, it is important to note that devices inserted into the bodily cavities must be sterile. Three ways to achieve this are: a) use a presterilized disposable device, b) use a sterile protective condom or sheath over a reusable device, or c) use a resterilizable reusable device. Common for rectal probes is choice (b) wherein the newly sheath-enclosed device is also cleaned between uses using chemicals. We will explain below that such sheaths can be made not to interfere with our sensor operation. We will depict such a sheath only in our later FIG. 4 but it will likely be used for all the depicted devices, at least for force/pressure sensing exams.

Typical dimensions for probe 1 in FIG. 1 are as follows. Dimension D may be on the order of 0.3-1.5 inches, with a dimension in the range of 0.6-1.0 inches being common. L3 may typically be at least 3-5 inches but may be as large as 6-9 inches. L4 may typically be hand-sized, which typically means 3-5 inches in length. L2 may typically be small, on the order of 1 to 2 inches or less.

In FIG. 1, the reader can see that both the force/pressure sensing array 2a and the temperature sensing array 2b are depicted as each having approximately 16 rows and 16 columns, comprising 256 total sub-element sensors each.

The force or pressure sensor array (not the temperature array to be discussed below) and control system may be that offered by Pressure Profile Systems, supra. The control box is their Model 2000 TactArray™ system, which can handle up to 256 sub-elements or channels and can fully scan such a sensor array at 20 hertz. It has a USB interface such that it can be driven by a PC using their provided PC-based software. The sensor itself may be a 16 by 16 or 256 sub-element (channel) sensor array connected to this control box. The three available TactArray™ sensor types are the (i) conformable, (ii) industrial and (iii) stretchable types. The conformable and stretchable sensors are available in 0-1 psi range or higher while the industrial version has a 0-3 psi minimum range. For our prostate application, we recommend either the conformable 256 channel sensor at 2 mm pitch (sub-element spacing) or the industrial 256 channel at 2 mm pitch, the former with 0-1 psi and the latter with 0-3 psi ranges. A higher number of channels than the above 256 allows for a bigger sensor array at the same 2 mm or so pitch pixel spacing. The conformable sensor is also available with 0-3 psi range if desired. These ranges are useful for prostate loading. Higher ranges may also be useable, for example up to 15 psi max, as the software allows for a scaling of the sensitivity up or down. We note that the conformable sensor is wrappable around our probes as we depict in the Figures. Although the stretchable sensor is highly compliant, it is currently available in 10 mm pitch, which is coarse for our application. The sensor is shown adhered or otherwise clamped to the probe body 1 permanently or temporarily.

It will be noted that the above sensors can be mounted to curved surfaces such as our diameter D and can be “read-out” at 20 hertz. We prefer a force/pressure sensitivity range of 0-1 to 0-3 psi and sub-element spacing or pitch of about 2 millimeters. Other models available from PPS offer a higher number of sub-elements with higher pressure ranges, such as the mentioned 0-15 psi. Generally the full sensor array will have N by M sub-elements and be of a size large enough to get a force (or temperature) map of the prostate. Typically, this would be at least as large as a fingertip (e.g. 0.25 inch square approx) up to an inch or so wide by one or more inches long. The idea here is that the sensor array is preferably at least fingertip size and is even more preferably bigger than that such that it covers the range over which a finger would have been laterally scanned (generally in the x-z plane but on the somewhat curved prostate-adjacent rectal roof surface) in the prior art. We also include in the scope of the invention sensor arrays smaller than that wherein larger areas (than the array) can be mapped by dragging or twisting to cover the complete test tissue site. It will be noted that the width of the arrays in FIG. 1 (in Z direction) are wrapped around the curved surface of probe 1. Thus, in FIG. 1, the probe sensor region has one radius and is cylindrical in nature. We also include compound curvature of a sensor in the scope of the invention.

In FIG. 1 we also depict in phantom item(s) 4a/4b that comprise additional sensors that provide additional information relating to the macroscopic state of position or motion of device 1. These might be, for example, a rotational tiltmeter such that the probe may be rotated known amounts around the X-axis or may have data readout at known angular increments around one or more axes. The sensor may also be a combination of gyroscopes or accelerometers such as of the MEMs variety. The most common sensors such as 4a or 4b would be tilt or rotation sensors and three-dimensional magnetic spatial tracking sensors. Alternatively, or in addition, an item 4a or 4b could be an inventive vibrator means to be discussed below.

So in order to perform an exam, device 1 may typically be covered by a thin conforming condom or sheath (not shown in FIG. 1), lubricated with a gel, and inserted in the rectum by the examining clinician as he/she holds the handle portion 3, at least during the step of insertion. For a force or pressure mapping exam, the clinician may manipulate the force/pressure sensor array 2a upwards (by lifting and/or tilting in the X-Y plane, for example) against the prostate. Ideally, the force/pressure array is large enough that it detects the pressure or force footprint of the enlarged prostate and/or prostate tumors. The forces imposed on device 1 sensor 2a by such BPH conditions and/or tumors will typically be higher than those applied by adjacent rectal wall tissues not backed by the prostate. In this manner, the raised or hard-spots are being felt by our detector rather than by the finger as in the prior art.

Although for simplicity we have shown in FIG. 1 the device having a constant insertable diameter D, we will show in later Figures a sculptured insertion surface which offers some detectability and sensitivity advantages once inserted.

Device 1 may be externally powered as by cord 4 or may have its own battery or power source therein (not shown) and even possibly have a wireless data connection. If batteries are used, it is preferred to utilize rechargeable batteries and to “park” the device 1 in a recharger (not shown) between uses. At any rate, FIG. 1 depicts a preferred approach wherein a cable/lumen 4 is connected or connectable (at least temporarily) to our device 1. If there is no wireless data connectivity provided, then-cable 4 may at least carry sensed pressure/force data back to a display/computation means such as a laptop or computer graphics display. Alternatively, sensed force data may be recorded in-situ and downloaded from the device after the device is removed from the patient. Alternatively, the display and/or computation means may be mounted on or in the device itself (not shown). A battery or fuel cell may allow for unplugged operation, particularly if the battery were occasionally recharged (with or without its removal).

Thus, included in our inventive scope is having the display and/or computational means one or both of separate from device 1 and possibly connected by cable (shown), or integrated inside of or upon device 1 (not shown). The computational or control means may operate the pressure/temperature sensors(s) 2a/2b and any position/orientation sensors 4a/4b and create the desired clinician output such as a force/pressure map or temperature map of a prostate or portion thereof. Some electronic logic, switching and powering circuitry may typically also be provided. It is preferable to provide switching circuitry (sub-element switching) in the form of an integrated circuit in the probe 1 to minimize the number of wires in any umbilical cable 4.

We note that the computation means (not shown) may simply advise the clinician as to its conclusion as to whether the sensed data indicates a problem or not. This could, for example, be done as by an audible tone or an animated or colorized graphic or warning light. It is not necessarily required to provide the examining clinician with full pressure/force maps or temperature maps at exam time or even thereafter. These may be reduced to a go/no-go flag or tone instead (or in addition). The sensed data may also be uploaded to a network and/or stored on the probe or an attached memory media. Note that a remote computation means and/or interpreting clinician may compute upon the data or analyze it remotely from the examination site at the time of the exam or at a later time. In a preferred approach, the actual sensed data maps corrected for spatial location if necessary will be stored in memory if later required or useful for any reason.

It will be noted in FIG. 1 that if the clinician rotates the device 1 around the X-axis by 180 degrees (X_{θ}), he/she can bring either the force/pressure sensor 2a or temperature sensor 2b into contact with the rectal roof-wall adjacent the prostate. In the shown embodiment of FIG. 1, sensor 2a is a force/pressure detection array and sensor 2b is a temperature detection array. In other implementations, one may choose to have two different opposed force/pressure sensors, for example, or two different temperature sensors, or only one sensor of a given type on one face or fully enwrapping. Multiple sensor arrays of the same type (temperature or pressure/force) may have different force/temperature ranges or areal sub-element densities. We also include in the scope of our invention having two sensors overlaying or being interdigitated with each other such that both force/pressure and temperature maps can be obtained of the same region with probe contact. We also anticipate the use of dual sensors whose sub-elements can measure both force/pressure and temperature.

The present inventors note that mapping of the force/pressure (or temperature) may be done as by electronic switching between sub-elements in a sensor array parked stationarily against the tissues. Alternatively, or in addition, mapping may be done by some amount of physical scanning of the probe itself by the clinician. In the latter approach, for example, one could have a single row of force (or temperature) sensor sub-elements along the X-axis and rotate these around the X-axis by twisting the probe 1 using the handle 3. We include in the scope of the invention the use of various means (such as 4a, 4b to be discussed below) to monitor such physical translations and/or rotations such that a properly motion-scaled map is produced.

Moving now to FIG. 2, we see a device 1 inserted into a rectal cavity of a patient and situated underneath a prostate gland. Specifically, device 1 is depicted inserted through the anal sphincter 7 into a rectal cavity or rectum 8 in a patient or test-subject 6. Above (in this view) the inserted probe 1, we see the prostate gland 9, which is also depicted as having two tumors 9a and 9b therein. The prostate gland 9 is shown as being approximately distance t behind the rectal upper wall, which is typically on the order of one or a few millimeters. Again, we see the inserted probe's coordinate system.

Note in FIG. 2 that we have, unlike FIG. 1, the sensor arrays mounted on compoundly curved surfaces rather than on simple cylindrical surfaces. In FIG. 2, we show a radius R which offers convex curvature to the sensors along the X-dimension. In the Y-Z plane the probe cross-sections are of a variable diameter D within length L1 with the largest in the middle of length L1. It can readily be seen that in the FIG. 2 insertion-state depicted we actually have some free rectal space underneath the probe 1 because the probe is being lifted or twisted upwards to contact the prostate. The radius R improves the sensitivity of detecting tumors 9a and 9b by reducing sensor loading by tissues adjacent but not part of prostate gland 9.

We anticipate at least two methods of applying the probe 1 to the tissue for mapping. The first is purely manual in nature as depicted wherein all probe forces are provided by the clinician's hand, the reacting or enveloping anatomy, and by any elastic characteristic (if any) of the probe itself. It will be noted that a probe parked against the prostate could provide a “static” force map even with the practitioner's hand removed from the probe handle 3; however, we expect some amount of physical hand scanning and therefore also some amount of positive hand loading being applied to the prostate 9 by the practitioner via the probe body.

The second method, which may typically be used together with the first manual method (but may also be used alone), involves manipulation-assistive mechanisms (not shown). The first and simplest of these may be an inflatable (e.g., saline) balloon situated on at least one probe surface such that its water-pressurization urges the probe 1 against/toward the tissue to be mapped. Such an inflatable balloon might or might not be attached to the probe while inside the rectum, but in any case, while the inflated balloon is providing the favorable sideways urging, it may at least be physically abutted against the probe. In FIG. 2, the balloon (not shown) may likely be substantially inflated in the shown open rectal space (or to create that open space by balloon-filling) underneath the shown device 1, thus urging the device or probe upwards. As will be discussed below, the assistive manipulation forces that move the entire probe (or at least the sensor portion) may provide for static or dynamic time-changing forces. An advantage of assistive loading, such as water-bag loading, is that the forces applied can be highly reproducible, assuming the fluid pressure is controlled in some manner.

Yet another manipulation-assistive mechanism is to provide a vibrator or impulse generator in probe 1. In this manner, the probe body may be applied to the tissue with both a static average force and a superimposed oscillating force. It will be seen below that the oscillating or dynamic force can improve detection sensitivity. The oscillating force component may even be larger than the static force component, and within the inventive scope is the physical contact between probe 1 and prostate 9 being constant or periodic. Typically, a static minimum preload is preferred, as provided by the initial probe 1 fit for example, just to assure good prostate 9 contact during all phases of motion scanning.

In general, at least the force/pressure sensor arrays (such as sensor array 2a) are themselves mounted upon the probe surface in a rigid manner, meaning that their underlying probe foundation is preferably rigid (or more rigid) when compared to the hardness of the adjacent sensed bodily tissues. This assures that any variation in localized static or dynamic hardness or pliability of the test tissues 9, 9a, 9b causes an appreciable corresponding static or dynamic pressure localized variation upon sensor 2a instead of a pressure-neutralizing or pressure-reducing conformational deformation of opposed tissue and sensor surface shapes. This does not necessarily require a completely rigid sensor; it requires a sensor rigidity harder than that of the sensed tissue, preferably a few times harder at least.

Another method of providing a manipulation assistive mechanism is to have the patient bounce on his feet, cough or attempt a simulated bowel movement. All of these will dynamically change the sensor pressure loading, even with the probe 1 not being externally manipulated. This bounce or bowel movement loading method works quite well in combination with the above-mentioned inflated saline-balloon static preload.

Included in the scope of our invention is the use of multiple-sized probes 1 or probes having exchangeable sensors or sensor positions. Also included in the scope of the invention are one or more portions of the probe or sheath being disposable, including just the wrappable sensor(s) 2a and/or 2b.

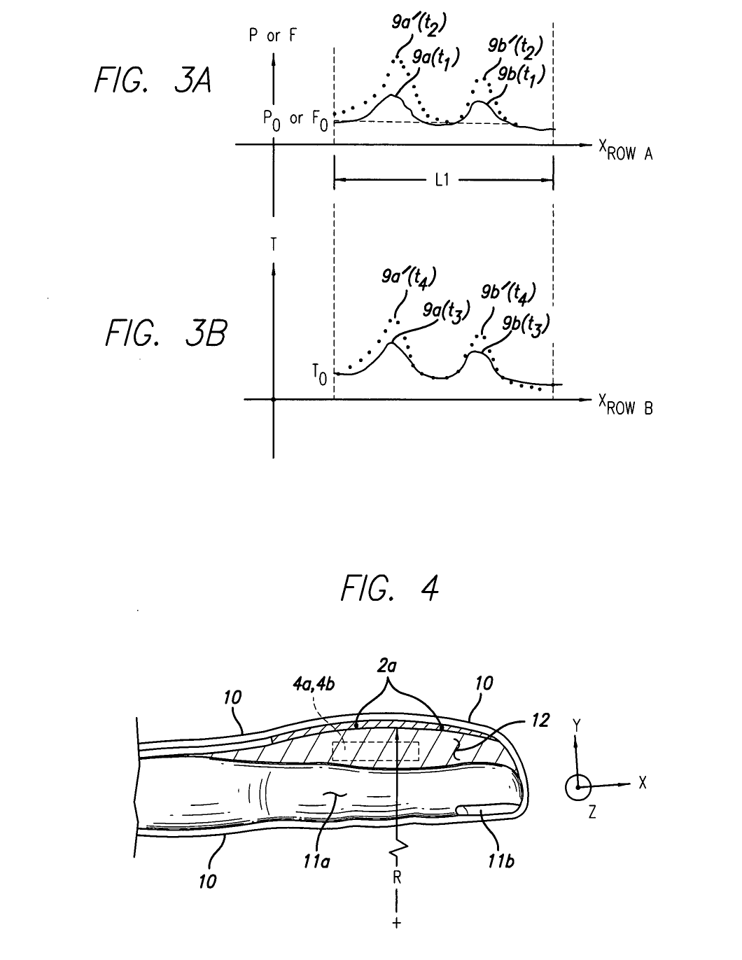

Before we discuss dynamic loading in depth in FIG. 3, we will first jump to FIG. 4 to describe a finger-mounted embodiment of the invention. In FIG. 4, we see the clinician's finger 11a having a fingernail 11b. Upon his/her finger 11a is mounted or placed an inventive force/pressure (and/or temperature) sensor 2a situated upon a rigid or semi-rigid substrate or foundation material 12. Typically, foundation material 12 is preferably harder or less deformable than any or the target tissues, at least during mapping of the tissues 9, 9a, 9b. Again, this is to maximize sensed localized force/pressure (or temperature) anomalies. Typically, foundation material 12 may be generally fitted to or conformal to the clinician's finger 11a. To achieve this, foundation 12 may have a deformable portion adjacent the finger (not shown), but the foundation portion adjacent the target tissues would still be relatively rigid (vs. tissues) as described above.

It will be noted also in FIG. 4 that we depict a snugly fitting surgical glove 10 which may also serve to help hold the device 12/2a upon the clinician's finger 11a. Such a thin stretched surgical glove is thin enough not to interfere with our force mapping. We may easily subtract any pressure forces the glove material applies upon sensor 2a such as by zeroing the array just before mapping. A critical note here is that we wish to choose a sensor array 2a that has enough force-range to capture all of the forces applied by the tissues and the glove. This may typically call for a sensor array capable of measuring 0-3 psi or so. Sensor arrays with much higher detection ranges (say 300 psi) might not be accurate enough at our low force ranges. Our mentioned preferred capacitive force-sensing array is available in such ranges from a few psi total range to hundreds of psi total range. Our success has been with sensors having for 0-1 psi range to sensors having 0-15 psi range since, as mentioned, the sensitivity of the sensor can be electronically adjusted in software.

The finger-mounted device 12/2a of FIG. 4 may be disposable in its entirety and may come with its own glove, preattached or otherwise. If any portion is saved, it would likely be the foundation or backer portion 12, as it might favorably be custom fitted or otherwise fitted or molded to the clinician as by it including a deformable or thermodeformable material adjacent the practitioner's finger surface. The clinician would still likely utilize a lubricant with the finger-mounted device of FIG. 4 to aid insertion.

Included in our inventive scope is a probe that is inserted and left in the rectum for a measuring period at least partly during which the clinician does not have to be manipulating or forcing (or even touching) the probe. This variation ideally utilizes probes that have saline-inflatable forcing balloons as previously discussed, as such a balloon can be inflated and left inflated for many minutes if desired. In an extreme example of the invention, the probe may be left in for hours and may allow the sphincter to be closed such that there are no protruding wires or handles. Such a device may likely also be or enable a connected recording device.

We also include in our inventive scope a dynamically applied tissue/sensor force being one or more due to natural dynamic bodily processes such as breathing, perfusion, bowel movements or urination and we note again that sensed dynamic forces may be superimposed upon static forces if the static forces are not subtracted or zeroed. The static forces do not necessarily have to be zeroed. Tumors and BPH enlargement can be detected even without such zeroing.

The next subject for discussion is dynamic forces vs. static forces. In particular, let us proceed to FIGS. 3A-3B now. In FIG. 3A, we see a plot or graph of pressure or force along one row “A” of a force or pressure sensor 2a running along the X-axis. It will be noted that we receive readings over the length of the sensor L1 as expected, say from something like 15-50 data points in that row, for example. In FIG. 3A, there are two plots shown on one graph, that of data 9a(t1), 9b(t1) and that of 9a′(t2), 9b′(t2). First, recall that 9a and 9b are the tumor sites depicted in FIG. 2 prostate gland 9. Pressure or force plot 9a(t1), 9b(t1) is sampled at time t1, whereas pressure or force plot 9a′(t2), 9b′(t2) is sampled at time t2, so these are two different force recordings taken at different times t1 and t2 but taken at substantially the same position or X-range where tumors 9a and 9b are located.

The peaks correspond to high-pressure (or different pressure) points adjacent the tumors 9a and 9b. Typically, prostate tumors 9a, 9b are harder than their surrounding tissues and thus, like pieces of fruit in a Jell-O® dessert, can be felt as lumps from the surface. The solid-line plot of FIG. 3A corresponding to the first dataset from time 1 (t1) is static data, meaning that the probe and tissues are substantially stationary with respect to each other. This would be the case when the probe is inserted and a saline balloon is inflated and the clinician stops manipulating it or substantially holds it steady. In this condition, a static force map is imposed upon the force/pressure sensor 2a as the inflation balloon urges the sensor 2a against the prostate anatomy 9/9a/9b. As expected, higher peak pressures during this first test at time t1 can be seen at the tumor 9a and 9b locations along the X-axis.

Now let us move to the second plot in FIG. 3A, that of 9a′(t2), 9b′(t2) sampled, for example, at a later time t2. This is a dynamic plot rather than a static plot. What this means is that there is tissue/probe relative motion during the sampling period of the data at t2. The sensor 2a has moved toward the prostate, therefore causing all the mapped forces from t1 including those at tumors 9a/9b to be higher now at t2. In such a case, the different “feel” of the buried tumors can be amplified as by having the prostate dynamically deforming such that the tumors move out-of-phase with the overall prostate organ. Such dynamic motions may be caused such as by suddenly moving the probe 1 while inserted, by vibrating the probe and/or prostate, or by having the patient bounce on his feet or simulate a bowel movement, for example. Another mechanism for such force amplification is that tumors may have different mass-density than surrounding healthy tissues such that they demonstrate different inertial reactions to motion changes. Another is that tumors have different dynamic stiffnesses and deformation rates such that they again react differently than healthy surrounding tissues. In our dynamic approach, the tumor will always react differently than the healthy tissues to a deformation or motion, causing an at least transient pressure map having amplified features. We explicitly note that such different tumor mechanical reactions, depending on the time-phase, will either amplify or diminish the pressure or force peaks shown in FIG. 3A. Thus, it is desirable to be able to sample the force readings at various times relative to an applied tissue motion in order to sample at least at the time of maximal loads or peak sizes and preferably at smaller loads or at the nominal static load. We have shown positive pressure or force peaks in FIG. 3A, meaning that the tumors 9a, 9b at moment t1 and t2 have higher applied contact pressures or forces compared to their adjacent healthy tissue, which only applies the lower pressure or force P0 or F0 depicted. In any event, these are all positive forces or pressures. In some cases, the dynamic time-phased sampling of force data can be chosen such that the “peaks” are actually lower pressure dips or troughs and the background healthy tissue pressure P0/F0 is actually higher or more positive. In any event, the pressure/force contrast will be observed and maximized with dynamic loading.

We explicitly note that by the pressure or force data being sampled we mean that at least one pressure or force reading is taken from one or more sensing sub-elements. Since each such reading takes some time, it takes a finite period of time to sample the whole array (or less time to sample a single row, for example). Thus, while doing such sampling, it is beneficial, if utilizing dynamic sensing vs. static, to note or control the time-phase of the probe or tissue motion excitation relative to that of the reading of particular sensing sub-elements. As an example, the cited preferred force sensor samples the entire array at about 20 hertz or 20 times per second. Thus, if we were also exciting or vibrating tissues at 20 hertz, then the dynamic loading would go through a full 360 degree phase cycle during one or a few such array reading periods. One might, in order to reduce the data recorded, desire to sample sub-elements only at desired time-points in mechanical excitation phase (for example, the phase point of maximal loading) or to “tag” the readings with the excitation phase at the time of reading the sub-element. Both of these approaches, if one desires to have all sub-element readings at a particular mechanical excitation phase point in time, require that the tissue/probe be mechanically excited with a period as for vibratory motion and readings taken over many such vibration cycles.

Particularly beneficial to the invention and its dynamic pressure/force detection method is oscillating the prostate and its contents (tumors, etc.) at a natural frequency of that organ or of its tumors therein. By doing this with an adjustable period vibrator and sampling only the maximal pressure readings at each array point, one can maximize the dynamic amplification of the desired pressure or force footprint. This increases the sensitivity of tumor detection.

So now we shall return to the earlier mentioned motion/spatial and/or vibrational means 4a, 4b of FIGS. 1, 2, and 4. First, this means may be a vibrator 4a to provide the dynamic artificial tissue/probe forcing excitation described above. The period of the vibration cycle may be adjustable and may even be automatically scanned through one or more frequency ranges automatically looking for maximized (resonant or anti-resonant) tumor pressure footprints. Applied vibration is intended to, at least momentarily, enhance a pressure fingerprint or contrast of the tumors. Typically, the vibration direction may be normal to the probe surface and in the Y directions or Y-X plane. This may comprise probe lateral translation and/or probe angulation or bending. However, it should be noted that a curved sensor surface such as those shown in FIGS. 2 and 4 may apply some normal motion components to adjacent tissues even if the probe is vibrated along its own X axis due to the effect of the ramp-shaped sensor surface. In any event, it is not desired to abrade the rectal wall during such vibration; thus, a normally-loaded non-sliding dynamic forcing is preferred. Such dynamic loading does not prevent manual lateral scanning of the probe and may even enhance it as it reduces sliding friction.

Rather than continuous multicycle vibration, we also include in the scope single impulses as provided manually or by a probe-embedded or probe-attached impulse generator 4a. In some applications, impulses, if not vibrations, may be applied using a reversibly cyclically inflatable or pulse-inflated saline bag (not shown), for example.

Means 4b may be a position, orientation or acceleration sensor such as a MEMs sensor or tilt/rotation sensor. The purpose of this sensor or sensors could be several including the following: a) to assure a desired dynamic excitation of a vibratory or impulse type in terms of period or amplitude, b) to assure a probe orientation relative to gravity or relative to an external magnetic position/orientation sensor assuming the patient is stationary, c) to measure a probe dynamic deformation or displacement as by vibratory bending, angulation or translation of the probe, d) to measure a static probe load which also causes probe bending, and/or e) to measure a probe rotation, angulation or translation position relative to an inertial reference system or relative to an external reference field such as a magnetic positioning field.

We have shown in FIGS. 1 and 2 the insertable probe portion being generally rotationally symmetric or being a body of revolution about the X-axis. However, in FIG. 4, the finger-mounted probe depicted therein is likely not rotationally symmetric nor a body of revolution about the X-axis. The present inventors anticipate the ideal force/pressure sensor array-shape in three dimensions will be more like that of FIG. 4 but some amount of “roundness” or rounding is desired for comfortable insertion. Thus, a typical probe product may likely have curved convex sensor surfaces but also have tighter curves in probe regions away from the sensor(s) on the circumference. The present inventors include in the scope of their invention a probe having an adjustable curvature sensor (preferably adjustable or selectable one or both of before or after insertion) or having a probe whose sensor region is differently curved or shaped than adjacent nonsensor regions. The present inventors also include in the scope of their invention a sensor that can be urged against the tissue to be mapped separately from the foundation probe as by inflation of a lifting mechanism to bodily lift the self-rigidized sensor array toward the adjacent tissue by “pushing off” from the foundation probe body. The present inventors also include the use of a mechanical sensor cover that overlays the sensor until after comfortable probe insertion and is retracted after probe insertion to expose a curved sensing array which would have been too uncomfortable to itself insert while exposed. Finally, we also include the option of a deformable sensor which can be set to a desired shape or curvature before or even after insertion.

Position sensing means 4b may be utilized to trigger array or array 2a/2b sub-element reading events. As an example, in one application we may have the clinician manually rotate the probe about the X axis slowly. The rotation or angle sensor 4b may trigger reading of a single-row pressure/force sensor 2a as that sensor row passes desired increment angles. In this manner, one obtains two dimensional information maps using only a one dimensional linear force sensor. The same strategy may be applied to manual translation along the X-axis using a single row sensor wrapped around the probe in the Y-Z plane. Note that this automatic triggering scheme assures that data is sampled at equal angular increments despite manual variations in the angle change.

In any event, we include in the scope of our invention the synchronization of reading sensors or sensor sub-elements in coordination with probe motions or any other type of mechanical tissue or probe excitations, such as the patient bouncing on his feet. Synchronization of a vibratory driver 4a may also be done in coordination with sensor reading.

Moving now beyond the inventive palpation (force/pressure mapping or detection) improvements, let us now focus on our before-mentioned sensor 2b, which is a temperature sensor. It is a long-known fact that tumors have slightly higher temperatures than surrounding healthy tissues in the matter of breast cancer and brain cancer. The same is true of prostate cancer; however, it has been difficult to obtain thermal maps of the prostate, and the present inventors have not found any reference to anyone trying to obtain such thermal maps in a non-invasive (non-prostate penetrating) manner. In any event, let us use FIG. 2 in explaining this aspect of the invention.

In FIG. 2, we see two tumors 9a and 9b in prostate gland 9. Also, imagine the probe 1 rotated 180 degrees about the X-axis such that the thermal sensor array 2b is then facing the prostate 9. Tumors themselves typically run about 1.5-2.5 degrees C. warmer than their non-immediate surroundings. Thus, the closer one is to a tumor, the larger the temperature peak one will see indicative of the nearby underlying tumor. Conversely, the deeper the tumor is buried, the more subtle will be the temperature peak caused by the tumor at the now more remote detection surface. Fortunately, many prostate tumors form as shown in FIG. 2 wherein the tumors 9a and 9b are situated near or adjacent the rectal cavity 8. This therefore means that these tumors should present thermal patterns or temperature peaks on the interior rectal upper wall against which our temperature sensor 2b sits.

Long experience with thermography in finding breast cancer has demonstrated that the thermal signature of underlying tumors can be enhanced by what is called the “cold challenge”. This is where the patient's vasculature is constricted by having them dip their hands (or feet) in cold water. Since tumor vasculature does not thermally vasoconstrict, what this does is suppress thermal signatures from vasculature unrelated to the tumor, therefore improving the signal/noise ratio of the real tumor thermal signal. The same effect can be had by administering a vasoconstrictive drug or medicament. Thus, we include the probe having a cooling or heating feature for manipulating tissue temperature or bloodflow and attempting to cause selective constriction of non-tumor vasculature.

Breast cancer thermography experience has also taught that one can enhance the underlying tumor thermal signature seen at the surface by cooling the surface as by blowing cool air on it or spraying evaporative liquids on it. Such surface-applied cooling of the rectal wall is also in the scope of the present invention.

It is now appropriate to discuss the various means of providing thermal sensor or sensor-array 2b because different thermal sensor-types demand different means of coupling to the tissue being examined. We shall group these into four main categories as follows:
1. Non-Contact Optical Thermography: This is basically air-standoff mid-infrared (MIR) thermography wherein the observed infrared signal in the mid-IR range emanates from the top 100 microns or so of exposed MIR emitting tissue and one typically observes surface patterns or gradients of color representing temperature across the air gap. Air is MIR transparent; however, saline is not. Thus, one cannot look through a significant saline thickness. The observed thermal gradients are due to surface vasculature and underlying vasculature and tumors, for example. The key here is that there needs to be a standoff distance between the IR camera or sensor (or its IR window or lens) and the tissue surface, the standoff gap usually occupied by room air or gas which is highly IR-transparent over reasonable distances. Modern MIR detectors, whether single elements or arrays of sub-elements, can detect temperature differences as small as 0.01 Degrees C. at 60 hertz frame rates to provide a dynamic full image. A number of imaging enhancement techniques such as image-subtraction is known for the breast application. The air gap only needs to be non-zero, i.e., some gap exists that is finite and nonzero. Generally, one may size the air gap to get the desired tissue field of view or to be at a safe and non-obstructing working distance.
2. Contact Optical Thermography: This is the inventors' novel variation of prior art thermography (above) wherein the standoff gap, usually filled with room air or gas, is instead occupied by a mid-infrared MIR (if not also visible and/or near-infrared NIR) transparent window material such as calcium fluoride, an excellent IR window material over those broad wavelength ranges. The present inventors have a separate patent application pending for the general application of this “contacting IR window” approach to organs such as breasts containing cancer or heat-producing disease or abnormalities such as infections; see, e.g., application Ser. No. 11/706,120, filed Feb. 14, 2007. The IR-transparent window thermally and physically touches the tissue and may therefore be used to controllably inject or remove heat from the tissues as well as be used to compress the tissues to disrupt blood flow or bring underlying features such as tumors effectively closer to the observable surface. In essence, the transparent window material allows for far more accurate temperature manipulation than hand-dipping or spraying evaporative liquids or blowing cold air on tissues being examined. It will be noted that because the IR window is IR-transparent, one may observe all of this through the contacting window such that the target tissue can be flattened (or curved controllably) and surface temperature-controlled. The technique allows for rapid surface temperature changes of high uniformity, thereby bringing out subsurface IR details from tumors, etc. Explicitly noted in that filing is that one may utilize an IR transparent liquid or gel either as the window or to thermally or optically couple the window to the tissue. Thus, as a window coating film, a very thin layer of IR transparent liquid or gel may be used overlaid on a solid MIR window. It will also be noted that for such a thin MIR window coating, the IR attenuation can be finite and one still gets appreciable IR signal through it.

Both approaches (1) and (2) can implemented using a mid-infrared (MIR) sensitive sensor of linear or areal design with a protective IR window such as sapphire or quartz. In particular, preferred IR imaging devices are the linear CCD area image sensor S9972/S9973 series devices from Hamamatsu Photonics (Japan). In the non-contact optical mode, the sensor and its window may be displaced from the rectal wall by an air or gas gap. In the contact optical mode, the quartz or sapphire window may actually touch the tissues and preferably displace surface water or mucus or be coupled by the above-mentioned thin sufficiently-IR transparent gel. Because this sensor is a linear sensor, one may likely arrange its long dimension (about an inch) along the probe length and implement physical or hand-scanning in the other direction using a twisting or angulation of the probe. A MEMs inertial unit or a rotation/angle sensor 4a or 4b may sample data at fixed angular increments. A 2-D IR thermal sensor may alternatively be utilized as may a protective cover which that while inserted and wipes off moisture on any window.

Before proceeding further, we stress that when we say MIR, IR or NIR, we include the option of having more than one wavelength range capability. For example, many NIR image sensors can also image in the visible. Some imagers have multiple sensor chips in order to obtain images in more than one such wavelength regime. So when we say MIR or thermographic thermal imaging, it will be understood that either that sensor chip or additional sensor chips may be used to obtain images in other wavelength regimes. Two popular combinations may be a) visible and NIR, and b) visible and MIR. It is advantageous to utilize visible imaging to locate tissue regions of interest.
3. Contacting thermally coupled probes: These are the many types of single-point and array sensors requiring physically thermally-conducting contact to the tissue. These include thermocouples, thermistors, precision resistors, temperature sensitive diodes, liquid crystals, etc. Such devices are commonly provided as single element devices with two or three leadwires; however, many of these may be fabricated in known lithographic or micromachining manners as arrays of a linear or 2-D nature, including thermistors, diodes, and precision resistors. Usually, if a large array is involved, one provides a circuit switching means to scan the many sub-elements so as to minimize the number of external wires or leads required. It is not the point of the invention to teach known and available thermal sensor array fabrication schemes.
4. Direct Optical Subsurface Temperature Detection: This is another inventive technique herein where one places an optically active dye subsurface in or to the tissues to be investigated. This may be by injection, by intravenous delivery or by ingestion, for example. In any event, the dye is optically excited from the tissue surface and produces an optical response spectrum having its own peaks, typically different than the ingoing excitation wavelength(s) or spectrum. One or more of the outcoming excited spectral features has a monotonically changing wavelength or amplitude correlatable to temperature of the excited dye at that location. At least one of the ingoing or outcoming optical peaks is a NIR peak having reasonable tissue penetration despite scattering. The point here is that one may detect the thermally-shifted or modulated light or spectral peak(s) and know that a region having a certain elevated temperature is present in the subsurface. Ideally, both the incoming and outgoing wavelength(s) are NIR, giving reasonable penetration despite the scattering. This approach may also be used with a NIR transparent contacting window as standoff, for example, that may also isothermalize and vasoconstrict the exposed tissue surface. Included in the scope of dyes exhibiting thermo-optical spectral modulation are nanoparticles such as gold or gold-coated known quantum dots or nanoparticles as delivered in a solvent.

So for techniques 2, 3 and possibly 4, we may have direct contact of the sensor element or sub-element array with the tissue. For technique 1, we may have a gaseous standoff gap.

When we say IR transparent window material, we mean solid or liquids that act as MIR or NIR windows as necessary. It may also act as a lens, diffuser, collimator or diffraction grating, for example. When we say optically transparent gas, we mean any gas including air, CO₂, and bowel gas or even vacuum. A solid window may be a crystalline material such as calcium fluoride or it may be a bundle of optically fused or unfused IR-transparent fibers such as quartz or sapphire fibers. Such fiber bundles may provide magnification and routing of an infrared image. Also, the “window” may include an IR lens that focuses IR light such as upon a CMOS or CCD sensor or upon a germanium, gallium or other III-V based device. Any such window of the invention may also be employed, if useful, as a means to shape tissue or thermally manipulate tissue.

Typically, for maximal lateral temperature-delta sensitivity, when using a contacting method, one wishes to minimize lateral thermal conductivity of the sensor array itself because if it were appreciably thermally conductive, then it would itself laterally sink and obliterate the lateral surface thermal gradients one is trying to observe. Thus, one may implement arrays of thermistors, thermal resistors or diodes, for example, in thin silicon or semiconductor patterned islands on an oxide, ceramic or glass substrate or wafer. Note that it may also be beneficial to cool the probe body in order to enhance the temperature gradient of the adjacent prostate gland. One may also take measures to prevent mucous or other fluid from wetting the spaces between thermal-sensing sub-elements in order to prevent those fluids from thermally coupling adjacent thermosensor sub-elements.

The present inventors specifically include in the scope of their invention the use of an enveloping condom or sheath-like member that is designed to be appreciably IR transparent to allow such temperature measurements through such a temporary disposable cover. For the previous force or pressure-sensing version by itself, one does not necessarily require IR transparency. MIR and other IR transparent polymers are known, such as those used in home-security IR lighting systems.

The present inventors include in their temperature measurement or mapping approaches the use of liquid crystal temperature sensitive films or materials, which may be abutted to and thermally coupled to the rectal wall and optically observed for the telltale color patterns showing the thermal gradients. One may do this, for example, by having a liquid crystal (LC) coated sheet that is urged against the tissues from the probe head and that is optically observed from underneath by lights and a camera in the probe head. Included in that scope is the separate insertion of the LC medium and the subsequent imaging of it with a later-insertable camera or imaging means. Also included in this scope is the use of temperature sensing arrays that become colorized or opaque/transparent in accordance with temperature and can be observed after removal from the rectum. These are quite simple and do not require any sort of in-situ camera or imager other than the temperature recording film or paper. They may preferably be one-time use; however, many LC-based ones are reusable. An advantage of a one-time use recording paper or dye is that one does not have to worry about the readings changing as one removes it from the body.

As we mentioned, breast thermography images may be enhanced by causing vasoconstriction and/or tissue surface cooling. In our FIG. 3B, we show two temperature plots of the two tumors 9a and 9b in prostate 9. The solid line plot is a static plot with no enhancement measures taken, taken at a first time t3. The dotted-line plot is a static plot having enhanced temperature peaks relative to background because we have suppressed surface heat sources such as by cooling the rectal wall ceiling with cool air or cool water which is displaced and flushed to allow MIR imaging of the exposed rectal ceiling. This is taken at a later time t4. By static here we mean the probe is not moving relative to the tissue (unless that is required to develop the 2-D temperature map as by laterally scanning a linear-array temperature sensor). The notations 9a, 9a′, 9b, 9b′ have the same meanings as in FIG. 3A. So what we are doing in FIG. 3b is analogous to what is done on the breast using breast thermography; however, we believe it is novel for the prostate, particularly, because different and new equipment must be used.

Note that in our finger mounted approach of FIG. 4, if sensor 2a were a temperature sensor rather than a force sensor, then we may want spacer material 12 to be thermally insulating such that finger-heat does not interfere with the readings and the observable thermal gradients are not laterally smeared out or reduced by the heat of the practitioner's finger 11a.

Another unique aspect of the invention is what we will call ringdown of the prostate and its contents. By this we mean that the prostate is mechanically excited as taught as by an impulse or oscillatory cyclic wave. When the excitation is turned off, the anatomical parts have their vibrations decay as a function of their mechanical properties, including their mechanical lossiness. The present inventors expect that a ringdown signature of the tumors may be detected that is separately identifiable from that of the prostate and its surrounding encapsulating relatively healthy anatomy. The present inventors herein specifically propose that we may use one or both of our force/pressure sensors such as 2a or our motion sensors such as 4a to detect such ringdown spectral characteristics.

We note specifically that the resonant frequencies of such anatomical structures such as organs and tumors typically range from a few hertz to a few hundred hertz or more. In this case, sensor 4a will likely beneficially have broader detection bandwidth that force/pressure sensor 2a. The recommended force/pressure sensor 2a is scanned for readout at about 20 hertz so unless this number is varied (which it can be) one will only see ringdown features observable at that frequency or harmonics thereof. Thus, we recommend that sensor 4a be a MEMs-based sensitive accelerometer with built-in analog to digital conversion, the type made by companies such as Analog Devices. These have very broad frequency bandwidth for our purposes here. So in that case, one might map the tumors with a force and/or temperature sensor and also look at mechanical ringdown of the excited organ using one of the sensors 4a or 4b.

We mentioned at the start that the prostate can have at least two medical issues relevant to the diagnostic capabilities of the invention herein. The first has been the much-discussed tumors and detection thereof with both the force/pressure and temperature sensors.

The second is BPH more commonly known as prostate enlargement. It is known that this is evidenced by a downward bulging of the prostate into the rectal cavity. Therefore, when the inventive probe is inserted into the rectum, like the probing prior art finger, the probe will be forced downwards to follow the contours of the bulging prostate. Note that in inserting the probe to/from the inserted position (at least to if not past the prostate), the probe is forced to follow a curved trajectory as it is urged downward. Thus, we have two entirely different mechanisms useable to find tumors and/or enlargement (BPH) and we shall now list these.

Force/Pressure Mapping Mechanism: This can see localized high-pressure points as force concentrations on the force/pressure sensor array.

A hard tumor will create a hard spot detectable as at least a high pressure spot upon the probe while the probe is being loaded statically or dynamically against the prostate incorporating the tumors.
The prostate itself presents as a large bulge or lump (generally larger than a tumor in it). Therefore, when the probe is passed over the edges of the prostate, the increased pressure as these edges slide across the force sensor can be detected. Similarly, when the force probe is parked on the prostate, there is an increased pressure across its face, and that pressure is higher for enlarged prostates than for non-enlarged prostates.

It is generally desirable that the length of the force/pressure sensor be longer than the depth of the enlarged prostate exposed portion (behind the rectal wall) such that the entire prostate can be sampled relative to some adjacent tissues.

It will be noted that by having a database of prostate force/pressure and/or temperature maps, one may compare the new results with database results for representative prior patients or even for the same patient at an earlier inventive exam. We include in the scope of the invention the practitioner being informed of any such comparative or normalized result in any manner during or after the exam, with or without the patient still present. This showcases the intrinsic advantage of the technology, namely, the ability to make non-subjective quantifiable comparisons.

We note again that any force/pressure measurement may be carried out statically or dynamically, statically meaning the probe and organs are at rest (except for breathing (perhaps)/perfusion) and dynamically meaning we use the aforementioned various means of tissue/organ excitation/vibration or simulated patient bowel movements to amplify pressure differences. Dynamically can also mean that the probe is being slid (inwards or outwards) and that the dynamic act of the prostate/tumors (if any) being lifted/dropped/dragged by the moving probe causes temporary forces that would not exist statically or would be of lesser amplitude statically. We note also that any probe that is rotated about its own axis that is itself not a body of revolution will cause similar rotational dynamic displacements. Likewise, a probe having a curved sensor face in the shown X-Y plane will undergo dynamic forces upon sliding along the X-axis.

Temperature Mapping Mechanism: This can see variations in temperature known to correspond to tumors, for example. A local tumor in the prostate may be accompanied by a warm spot adjacent it evidenced on the rectal wall. Likewise, a prostate having tumor activity therein will, on average, run hotter in bulk than a prostate that is tumor-free. The excess heat is caused by tumor metabolic activity and/or tumor enhanced vasculature.
We described a number of surface temperature measurement devices including thermally contacting ones (thermocouples, thermistors, diodes, precision resistors, etc., inventive contacting IR thermography windows) and optical non-contacting ones such as standoff infrared thermography. We also described the standoff being a gas or being an IR transparent window or liquid material that may be rigid or flowable.
We described also a means to detect temperature under the tissue surface, typically using at least one NIR wavelength(s) (ingoing, returning, or both). An excitable dye, chromophore or nanoparticle may be used whose re-radiation spectrum correlates with local temperature in a known manner. In some such cases, the tissue itself may act as the excitable species.

Included in the scope of temperature measurement, in a manner similar to force/pressure mapping, is static and dynamic temperature measurements analogous to what has historically been done for breast thermography. Included therefore are the cooling or warming of the tissues using any cooling or warming means such as probe heaters/coolers, jets of warm/cool fluid or gas, radiant lamps etc. The patient may also be administered a drug or medicament which enhances one or both of force/pressure maps or temperature maps.

Included in our inventive scope is the creation and/or referencing of a database of prior patients' actual results or of modeled simulated results for any of the force/pressure or temperature maps, the ringdown behaviors, or for the correlations therebetween. The clinician may input patient information such as sex, weight, age, height, obesity-degree, etc., such that the particular patient's results may be compared, at some point, to the results of a large population of patients. We include the possibility of using any saline or liquid (or gas) inflation balloon or condom for purposes of quantifying the volume and/or overall expansive compliance of the rectal cavity. We expect that a correlation will be found between BPH degree and such compliance/volume behavior.

The clinician may receive the diagnostic data in one or more forms immediately (during probe insertion), upon probe withdrawal, or after the patient has left the exam room. Such data, or portions or reductions thereof, may be recorded or communicated over a wired or wireless network and may also or instead be stored onboard the probe itself. Another possibility is to have some of the data, or an indicator of it, annunciated by a probe-mounted or console mounted display or synthetic voice, for example. Note that the inventive probe may or may not require a control console, depending on whether battery powering is used and whether an off-board PC or computer is used to look at the data. One might also plug the probe into a docking station after use, the docking station providing one ro more or data extraction, mapping presentation, database comparison of this patient vs. others, probe sterilization/cleaning or probe recharging, for example.

The probe system may recommend or automatically implement a particular test sequence (e.g., a vibratory excitation scheme) and may prompt the clinician to manipulate the probe in a particular manner (insert, withdraw, rotate, angulate, warm, cool, mechanically excite, etc.). In a more complex implementation, the practitioner may insert the probe whereupon any sensor scanning (if any is needed) is done automatically.

Preferably, any force/pressure sensor array may be re-zeroed after any enwrapping condom or sheath is installed on the probe or if, for example, the force sensor is affected by temperature changes or the liquid-pressurization of an inflation balloon or sheath.

We have discussed the use of inflatable members useable to preload or dynamically-load the probe against one or more of the rectal cavity walls. (They may also be used, as mentioned, simply to measure volume and/or compliance of the rectal cavity.) One attractive means of implementing these is using saline and a hand-bulb that can be squeezed to cause saline pressurization. Preferably, a pressure indication means that directly or indirectly measures or infers and displays the pressure is utilized. The needed saline or other liquid might even come prepackaged with the balloon or sheath and include the tubing and/or inflation bulb and checkvalve.

Some preferred embodiments of our inflatable means are as follows:
(a) a saline or air-inflatable balloon, bladder or membrane that urges the probe against the prostate in an upward manner toward the rectal wall ceiling (upwards being toward that ceiling regardless of patient orientation);
(b) a saline or air inflatable balloon, bladder or membrane that delivers or removes heat from the anatomy to benefit a force/pressure or temperature test;
(c) a saline or air inflatable balloon, bladder or membrane which is cyclically inflated/deflated to produce a cyclic excitation force or pressure;
(d) a saline or air inflatable balloon, bladder or membrane that is cointegrated with a probe sheath or condom;
(e) a saline or air inflatable balloon, bladder or membrane that, in at least one uninflated or inflated state, allows for the detection of a force/pressure or temperature by a probe sensor in any manner;
(f) a disposable condom, sheath, balloon, bladder or membrane or combination disposable thereof;
(g) a saline or air inflatable balloon, bladder or membrane that also acts to seal the anal sphincter in any manner during any portion of insertion, testing or withdrawal;
(h) any balloon, bladder or membrane that itself incorporates any type of optical window for any type of IR, MIR, NIR or visible imaging; and
(i) any balloon, bladder or membrane that itself incorporates a force/pressure or temperature sensor, such as a capacitive force sensor array or an LCD temperature sensing film.

Regarding “balloons, bladders or membranes” we broadly mean any deformable or distensible body such as those typically inflated as by gases and liquids but also including those deformed by underlying or internal mechanisms. In most cases, however, the balloon, bladder or membrane may comprise a polymeric inflatable membrane. The membrane material may or may not be elastomeric or stretchable in some elastic or plastic manner. Generally, an elastomeric or plastically-deforming membrane or balloon that can assure tissue conformance without wrinkles is preferred. The balloon may be symmetrically or asymmetrically located or mounted with respect to the probe. It does not necessarily have to surround the probe diameter or be a body of revolution. A preferred embodiment has an inflatable or deformable balloon, bladder or membrane underneath the probe such that filling or inflating it lifts the probe's force (or temperature) sensor array into contact with the overlying prostate. The balloon may be inserted before or along with the diagnostic inventive probe. In one case, one may preinsert a balloon, inflate it to temporarily distort the anatomy or to precool the anatomy, for example, then insert the probe (with or without balloon removal) and monitor the physical and/or thermal relaxation. The balloon, bladder or membrane may have a hole or channel to receive the probe. The balloon, bladder or membrane may also be inflated outwardly from a recessed compartment in the probe body and even be deflated to allow probe removal. Any such balloon or membrane may be used to perform the earlier mentioned rectal cavity volume/compliance inflation test, with or without the probe being present during that test step.

Within the scope of “balloon” we include the use of the rectum itself as an inflatable or at least pressurizable cavity. This would be possible at least for momentary low applied pressures, for example, if the anal sphincter is sealed. One may also desire to provide an upstream seal (provided by the probe or by a separate seal means) between the colon and rectum. A balloon or membrane is preferred over trying to utilize the rectum itself as a balloon.

Like enlarged prostates and prostate tumors that present force or pressure anomalies to our inventive probe, we also expect the probe to be able to detect anal and rectal cancers using the same principle. For this purpose, the probe's sensors may be oriented in one or more directions other than or in addition to toward the prostate. Such a probe might benefit from a 360 degree wraparound sensor.

Additional Features

1) It is preferable that the sensor length be long enough to include within its length the pressure (or temperature) footprint of the entire prostate as well as of any prostate tumor therein. In this manner, the non-prostate adjacent tissues may serve as a reference surface for such measurements.

2) It is preferable that any contacting sensors or sensor arrays be preloaded against the tissues with a known total force or contact-pressure. This essentially normalizes readings from patient to patient and from exam to exam in a single patient.

3) It is preferable that at a controlled preload one records the entire force (and/or temperature) map, particularly peaks and minimums as well as slopes and gradients. Larger than normal peaks and high than normal gradients are frequently associated with abnormal enlarged or cancerous tissues.

4) It is preferable that the pressure/temperature mapping be done in two dimensions or directions that are generally orthogonal at least one point. By doing so, one may still utilize Cartesian, cylindrical or polar coordinate systems in mapping and computation.

5) The probe may be designed such that it can be completely inserted in the rectum, perhaps except for leadwires, such that the patient can move or squat and pre-sent even larger loads upon the probe. The load irregularities are further amplified by this behavior as they are for dynamic movements such as bouncing on one's heels. This approach may be less practical if the probe had a handle sticking out during patient movement. Such a probe may also be wireless and recording.

6) Normalization of the force/pressure maps and even of the temperature maps may be utilized to make data from one patient comparable to that from another or from a relevant patient population.

7) An elongated probe may have a shape that is not a body of revolution or extrusion such that by twisting or rotating it (or sliding it), tissue loading is changed by such probe movement and the force/temperature response is measured. Also, the long probe axis does not necessarily have to be straight; it may be curved to enhance insertion and/or compliance to the rectal ceiling.

8) One or more sensors may be integrated within or upon one or more balloons, sheaths, bladders or membranes.

9) A balloon, membrane, sheath or bladder does not necessarily have to be inflated or distorted; it may merely serve to isolate body fluids from the probe workings. In this case, it might preferably be snugly elastomerically fit over the probe to prevent wrinkles. At least the region over the sensor should remain unwrinkled.

10) Sensor arrays or probes may be offered in more than one size or in adjustable or selectable sizes. The sensor(s) may be mountable on the probe and may be disposable. The sensors may include their own backing portion.

11) A probe may include an imaging modality such as ultrasound imaging. It may also be designed to fit on an existing rectal ultrasound-exam probe.

12) The probe may also contain sensors such as electrical conductivity or electrical or magnetic permissivity/permittivity sensors known to be sensitive to sodium/potassium pumps at tumors.

13) The probe may, utilizing a dye, contrast agent, or targeted microbiological species or decorating nanoparticles to also image or detect cancerous tissues. The dye, etc., may be administered before or during the probe exam.

14) Chemicals, drugs or thermal exposures may be utilized together or separately from the lubricant gels or inflation liquids, which cause the hardness of tumors to be further accentuated or the heat-signature of a tumor to be further accentuated.

15) Any chemical, dye, contrast agent, drug or lubricant may be provided separately or as pre-coated on a sheath, membrane, bladder, balloon, probe or sensor itself. The probe and/or any balloon/membrane may dispense, be coated with or out-diffuse a useful medicament, contrast-agent, anesthetic, thermal or optical couplant or inflation medium, for example.

16) Prostatitis may also be detected using the invention, as that can present as an enlargement, swelling and/or abnormally warm organ.

17) Prostate enlargement may, for example, be determined at one or more total sensor preloads (or varying preloads) by any one or more of: a) looking at the load portion due to the prostate vs. adjacent non-prostate tissues, b) looking at the dynamic load portion due to the moving prostate, or c) looking at the prostate overall temperature with or without metabolic stress.

18) Prostatic tumors may be detected, for example, a) by noting hotspots on the thermal array, b) by noting high pressure spots on the pressure array, and/or c) by noting a correlation between a hot spot and a pressure anomaly.

19) Prostate and surrounding structures may be excited at one or more frequencies and one or more of various resonant frequencies and/or differences in ringdown behavior noted that correlate with one or both of prostate enlargement or prostate tumors. We include in our inventive scope the mechanical excitation of these tissues in any manner including using the probe or probe component or using a balloon, sheath, membrane or bladder that may be deformed/inflated/deflated at the required excitation rate. Excitations would likely be frequency-scanned so as not to miss resonances or anti-resonances.

20) The patient may be required to stand, stoop, bend-over, sit or lie down or even to abut his crotch/body against an external surface in order to optimize the mechanical behavior of an exciter or of a tissue loading mechanism. The exciter may even be external to the body.

21) The probe may include or be used with blood-detection chemicals or lights that detect blood in the stool.

22) The probe may be designed for home-use or use by a technician as opposed to requiring use by a clinician or doctor.

23) Any one or more of, for example, the entire probe, the sensors(s), or the balloon/sheath may be disposable.

24) The probe data may be normalized or referenced to a patient's body weight, age, height or other health parameter and/or with the use of any diet, drugs or surgeries (past or future) that might affect the expected data.

25) We anticipate the need to calibrate the probe periodically (pressure and/or temperature) and for this purpose we anticipate the use of a pressure-applying or temperature-applying calibration fixture or ambient. This may most accurately be done with an inflated membrane and/or a temperature bath, respectively. The use of a kit-provided inflatable balloon or membrane to serve this function is within the scope of the present invention.

26) We anticipate the zeroing-out of any mechanical preload such as due to a tightly conforming condom, sheath, membrane or bladder.

27) We anticipate that the probe and attached system may have the ability to capture particular parameters such as a maximum load or a maximum load gradient during one or both of a static or dynamic loading test.

28) We anticipate a multiuse sensor that is exchangeable when it goes out of useful calibration range for whatever reason, such as wear or damage, for example.

29) In order to accurately measure pressure or force maps using the sensor and to compare these maps to others, it is important to conduct the mechanics of the force/pressure map test reproducibly in a manner that maximizes the signal-to-noise ratio. We specifically prefer a scheme wherein the force/pressure sensor is mounted on a substrate that is less deformable than adjacent tissues (at least during actual measurement) and that is preferably of a reproducible shape. By less deformable we mean preferably at least twice as hard and more preferably at least 10 times as hard. This assures that anomalous hardness tissues cause full anomalous corresponding localized loads. The sensor surface is preferably flat to convex at least during measurement.

30) In a dynamic loading test, we anticipate a dynamic load may be developed or applied as by moving the probe from within or without the probe or by having a dynamically projecting member of the probe such as a foundation substrate that lifts the sensor up and down dynamically. This foundation may have a controlled mass and spring constant.

31) In order to accurately measure temperature and localized temperature anomalies, it is important that the temperature sensor have minimal thermal mass and be isolated from foreign heatsinks or sources. We include in the scope of our invention a temperature sensor that, as stated, is non-contact in nature (or operates across a thermally insulating gap) or has a small thermal mass and is thermally isolated from the probe body (unless the probe body is itself of a controlled temperature, wherein contact may be allowable).

32) Included in the scope of our invention is the use of a sensor that requires the use of a sheath such that the sensor is not damaged by bodily fluids and/or such that the sensor does not require sterilization.

33) Included in the scope of our invention is the use of a sensor wherein a sheath or condom provides or also provides at least some if not all needed fixation of the sensor(s) to the probe.

34) Included in the scope of our invention is the use of a probe wherein the clinician or user manually activates a tissue vibration or excitation as by hand inflation of a squeeze-bulb or tapping of the probe handle.

35) The inventive probe technology may also be used intra-operatively on various other bodily organs including, without limitation, the liver, kidney, lungs, heart, intestines, stomach, etc., and during such use, the probe may even be under blood or bodily fluid.

36) It is anticipated that the probe system may preferably be capable of reporting pressure or temperature differences from prior maps, from patient-population statistically determined maps, for a drug vs. no-drug map or for a loaded vs. unloaded map, the load being a load change applied to the probe or by the probe in any manner.

37) The force or pressure sensor may use, for example, capacitive, resistive or piezoelectric-sensor mechanisms of fiber-optic Bragg mechanisms.

We shall now make a list of some of the itemized configurations which we teach, by item number:

1) A prostate probe system for assessing one or both of BPH or prostate cancer comprising:

(a) a force or pressure sensor mounted on or in a rectally insertable probe and necessary means to power, connect to, switch and read the sensor;

(b) the force or pressure being employed to sample two or more rectal wall locations adjacent or juxtaposed to the prostate from the rectal wall during insertion or while inserted;

(c) the sensed data from the two or more location's forming a force-map, pressure-map or data array indicative of the patient's underlying prostate; and

(d) a comparison or computation means which, using at least the sensed two or more data points, computes or determines a degree of likely BPH enlargement or likely tumor presence;

(e) that information being at least one of recorded or reported during the exam; and at least one of

(1) an optional mechanical exciter integrated in or coupleable to the probe;

(2) an optional motion, deflection or inertial sensor integrated in or couple able to the probe;

(3) an optional deflectable or inflatable balloon, membrane or mechanism capable of applying a load or deflection to one or both of the probe or the anatomy; or

(4) an inflatable balloon or membrane used, at least in part, to measure a volume or compliance of any of a rectal wall or cavity or to heat or cool anatomy.

2) The prostate probe system of item 1 wherein any of the force or pressure readings are taken any of:

(a) while the patient and probe are essentially static or in mechanical equilibrium except for unavoidable perfusion and breathing motions;

(b) while the patient moves, simulates a bowel movement, or bounces his anatomy intentionally, some of the readings thereby possibly being dynamic or transient readings;

(c) while the clinician moves or manipulates the probe manually, or with the optional exciter, some of the readings thereby possibly being dynamic or transient readings;

(d) while the clinician moves or manipulates the probe or anatomy with the optional deflectable or inflatable balloon, membrane or mechanism, some of the readings thereby possibly being dynamic or transient readings;

(e) after the clinician moves or manipulates the probe or anatomy with the optional deflectable or inflatable balloon, membrane, mechanism or exciter, some of the readings thereby possibly being dynamic readings;

(f) by moving the probe or its sensor from location to location in any manner to take any of static, dynamic or transient readings; or

(g) readings are taken from two or more different locations by two or more corresponding sensor sub-elements located, at a given time, at those locations within a juxtaposed sensor array, some of those readings being at least one of static, dynamic or transient readings.

3) The prostate probe system of item 1 wherein the probe is or has at least one of:

(a) is finger mounted;

(b) is a standalone probe itself being insertable;

(c) is at least partially covered or wrapped in a condom, sheath or membrane while inserted;

(d) is at least partially contained-in or manipulated by an inflatable membrane, balloon or deflecting mechanism;

(e) is immersed in any inflating medium, gaseous or liquid-like, with or without a containment balloon or membrane, the medium introduced for any purpose,

(f) has a sensor array wrapped-upon or mounted to at least one surface portion or surface region;

(g) has a sensor array which is 1×n sub-elements in length, m×n sub-elements in areal size, includes a mechanically scannable sub-element or sub-element array; or

(h) has a sensor which can operate in timed-coordination or synchrony with any of 1) the operation of the optional exciter, 2) the clinician's manipulation of the probe, 3) the inflation or deflection of an inflatable balloon, membrane or mechanism.

4) The prostate probe system of item 1 wherein the optional motion, deflection or inertial sensor is used for one of more of:

(a) to detect the operation of an optional exciter;

(b) to control the operation of an optional exciter;

(c) to detect or control a clinicians manipulation of the probe;

(d) to detect or control a patients willful movement of the probe or its adjacent anatomy;

(e) to achieve a desired excitation frequency;

(f) to monitor or control a rotation, angulation or translation rate of the probe via the clinicians manual manipulation;

(g) to inform the force/pressure sensor or sub-element(s) thereof when or if to sample said forces or pressures;

(h) to determine or compute a degree of loading upon the probe by either the patients anatomy or the clinicians hand or finger; or

(i) to determine a static or dynamic load, force or pressure applied by the optional deflectable or inflateable balloon, membrane or mechanism.

5) The prostate probe system of item 1 wherein coordinate transformations are performed such that any two or more of a force/pressure sensor; an optional motion, deflection or inertial sensor; an optional mechanical exciter; an optional deflectable or inflatable balloon, membrane or mechanism; or a clinician manipulated probe-handle utilize or are referenced to a common computational coordinate system for ease of computation.

6) The prostate probe system of item 1 wherein any of:

(a) a sensor or sensor array is fabricated utilizing flex circuit technologies;

(b) a sensor or sensor array utilizes capacitive or resistive sub-element(s);

(c) a sensor or sensor array is read-out, fully or in-part, at a controlled rate;

(d) a sensor or sensor array has electrically or optically addressable sensor sub-elements;

(e) a sensor or sensor array is disposable;

(f) a sensor or sensor array is larger in at least one dimension than a tumor which might be detected;

(g) a sensor or sensor array is larger in at least one dimension than a prostate gland dimension which can be sensed at the rectal wall;

(h) any portion of a sensor or sensor array element or sub-element(s) is read as triggered or gated by a clock or by data from the optional motion, deflection or inertial sensor;

(i) maximum or minimum static, dynamic or transient force or pressure readings are any of detected, recorded or compared; or

(j) any of the force/pressure absolute values or spatial or time derivatives or slopes of such data or data-graphs are utilized in determining an extent of prostate enlargement or tumor-presence likelihood.

7) The prostate probe system of item 1 wherein a force or pressure sensing element, sub-element or array of such sub-elements is situated, held, clamped, tensioned-across, suctioned, adhered or otherwise mounted upon or to a foundation or backing material having stiffness or rigidity larger than that of the typical tissues being examined-the tissue force variation thereby being preserved for detection by avoiding desensitizing conformational relaxation of the sensor shape.

8) The prostate probe system of item 1 wherein a sensor element, sub-element or sub-element array has at least one curved dimension or plane of curvature as mounted on the probe or as manipulated by the probe that enhances the probe's ability to either of pressure-map or temperature-map a target anatomy.

9) The prostate probe system of item 8 wherein said at least one plane of curvature at least one of:

(a) generally conforms to a typical healthy anatomy;

(b) fits or conforms to a healthy anatomy in a manner presenting a substantially uniform or a substantially normal healthy force/pressure map;

(c) allows for a smooth or comfortable probe insertion or manipulation;

(d) optimizes sensitivity across a sensor dimension or direction; or

(e) attempts to account for known variable prostate shapes, sizes or hardnesses.

10) The prostate probe system of item 1 wherein a force/pressure sensor array has at least one radius in one plane which is substantially larger than that of the insertable probe body radius or finger radius.

11) The prostate probe system of item 1 wherein any of:

(a) only static force/pressure readings are taken;

(b) only dynamic or transient force/pressure readings are taken;

(c) both static and dynamic or transient force/pressure readings are taken;

(d) only maximum or minimum force/pressure readings are taken or are reported;

(e) one or more disease-likelihood, disease-degree or diagnostic parameters are computed using one or more static and/or dynamic force or pressure readings;

(f) one or more sensing elements or sub-elements takes readings at two or more times;

(g) two or more sensing elements or sub-elements at different locations are read at substantially the same or at different times; or

(h) which element or sub-element(s) is/are read is determined, at least in part, by a probe orientation, a known load on the probe, or a state of mechanical excitation of the probe and/or the anatomy.

12) The prostate probe system of item 1 wherein any of:

(a) the probe is powered by an internal energy storage means at least sometimes;

(b) the probe is powered by an external energy storage or source means at least sometimes;

(c) the probe is capable of using a rechargeable or reenergizable energy storage means; or

(d) the probe has any of a wired, wireless, data or fluid/gas lumen-connection to any of a support utility, console or to a network.

13) The probe system of item 1 wherein at least one measured, sensed, detected or saved said force or pressure reading at one or more sensor elements or sub-elements is at least one of:

(a) a substantially static force or pressure;

(b) a substantially dynamic force or pressure sensed during a static or dynamic mechanical loading or excitation of the probe or adjacent anatomy;

(c) a substantially transient force or pressure sensed after a removal-of or change-in a static or dynamic mechanical loading or excitation;

(d) a force or pressure on an upwards or increasing amplitude slope;

(e) a force or pressure on a downwards or decreasing amplitude slope;

(f) a force or pressure having a known time-phase relationship with a static, dynamic or transient loading or excitation;

(g) a force or pressure nearing or at a peak value or minimum value;

(h) a spatially or time-averaged force or pressure from one or more sensor elements or sub-elements, said elements not necessarily being adjacent ones;

(i) a force or pressure determined to be outside-of or inside-of a range related to a patient population; or

j) a force or pressure which has substantially settled to a constant value after a transient or waiting period.

14) A prostate probe system for assessing one or both of BPH or prostate cancer comprising:

(a) a temperature sensor mounted on or in a rectally insertable probe and necessary means to power, connect to, switch if necessary and read the sensor;

(b) the temperature sensor being employed to sample two or more rectal wall locations adjacent or juxtaposed to the prostate from the rectal wall during insertion or while inserted;

(c) the sensed data from the two or more location's forming a temperature-map or temperature data-array having relationship to the patients underlying prostate;

(d) a comparison or computation means which, using the sensed two or more temperature data points, computes or determines a degree of likely BPH enlargement or likely tumor presence; and

(e) that information being at least one of recorded or reported during the exam; and at least one of:

(1) an optional mechanical exciter integrated in or couple able to the probe;

(2) an optional motion, deflection or inertial sensor integrated in or couple able to the probe;

(3) an optional deflectable or inflatable balloon, membrane or mechanism capable of applying a load or deflection to one or both of the probe or the anatomy; or

(4) an optional means of injecting or removing heat from a tissue region of interest.

15) The prostate probe of item 14 wherein any one or more of:

(a) a temperature measurement or sensing event utilizes a thermally contacting sensing means including any of a thermocouple, thermistor, diode or precision resistor;

(b) a temperature measurement or sensing event utilizes any type of optical sensing means including mid or near infrared optical means;

(c) an optical temperature measurement or sensing means utilizes one or more of: a gaseous standoff gap; an optically transparent window standoff material of any solid or liquid-like type whether the window material contacts the tissue itself or not;

(d) a two- or three-dimensional array of temperature detection sub-elements is provided; or

(e) one or more temperature detection elements or sub-elements utilizes an optical component to achieve spatial scanning.

16) The prostate probe of item 14 wherein any one or more of:

(a) two temperatures taken at two different times are recorded, compared or reported;

(b) two temperatures taken at two different tissue locations are recorded, compared or recorded;

(c) a maximum or minimum temperature at a tissue location is recorded, compared or reported;

(d) an increasing or decreasing temperature at a tissue location is recorded, compared or reported;

(e) the slope of a temperature change at least one tissue-location or region of locations is computed, recorded, compared or reported; or

(f) a substantially static, dynamic or transient temperature or temperature change-rate is computed, recorded, compared or reported.

17) The prostate probe of item 14 wherein any of:

(a) substantially rectal wall surface temperatures are detected or measured;

(b) substantially underlying subsurface temperatures are detected or measured; or

(c) a tissue temperature can be manipulated favorably using a probe system or probe-related heated or cooling means, favorably meaning allowing for a better signal-to-noise ratio of the temperature signal being sought.

18) The prostate probe of item 14 wherein any of the listed optional features allows for any one or more of (a) spatial motion control or monitoring of the probe, (b) improved temperature accuracy or improved spatial accuracy of temperature patterns sampled from the anatomy, (c) determination or control of temperature sampling sites or locations, or d) triggering of temperature data taking at least one sensor sub-element.

19) The prostate probe of item 14 wherein the probe is one of (a) a finger-mounted probe, (b) a standalone probe which is itself insertable in the anatomy, (c) an at least in part disposable probe, (d) a probe which is protected during use by a sheath, membrane or condom that is arranged not to substantially interfere with temperature mapping, (e) a practitioner manipulatable probe, or (f) a probe which any of records or transmits data in a wired or wireless fashion.

20) A prostate probe of combined items 1 and 14 having both force/pressure detection capability and temperature detection capability, said probe also preferably allowing for one or more of:

(a) physical registration of the force/pressure data and the temperature data if both data types are taken;

(b) sampling or detection of force/pressure data and temperature data from a combined or interdigitated sensor or sensor(s);

(c) taking of either or both data types in any desired sequential, parallel or time-interleaved sequence; and

(d) the ability to detect an undesirable tissue condition evidenced both by a pressure/force anomaly and a temperature anomaly.

21) The combined prostate probe system of item 20 wherein said probe is one or more of:

(a) finger mounted;

(b) a standalone probe itself capable of being inserted;

(c) has at least one exchangeable or disposable sensor of at least one type

(d) has exchangeable or disposable sensors of both types;

(e) utilizes a reusable sensor or handle, reuseable meaning used on two or more patients;

(f) during measurement is covered by a sheath, condom or membrane which is arranged not to substantially interfere with said pressure/force or temperature measurement(s);

(g) has a force/pressure sensor in one probe region and a temperature sensor in a second probe region, the two regions preferably being opposed regions presentable to target tissues via simple rotation of the probe;

(h) is wipeable or immersable in a liquid, gaseous or plasma sterilant without a covering condom, membrane or sheath installed;

(i) contains or is connectable to a power source; or

j) contains or is connected to a wired or wireless data network or a data recording means.

22) Any of the prostate probes herein wherein a patient is examined at two or more points in time, including even wherein said two or more times comprise two or more sequential exams done before and after a therapy is delivered, the results from at least some exams being compared to each other or to a those of a larger population.

23) Any of the prostate probes herein wherein the exam is performed by any of a doctor, clinician, technician or patient himself.

24) Any of the prostate probes herein wherein the patient receives a medicament or drug which enhances the sought signal of a prostate tissue abnormality, whether that signal be a pressure/force signal or a temperature signal.

25) Any of the prostate probes herein wherein the patient has his tissues manipulated or excited in a mechanical way to enhance the sought signal of a prostate tissue abnormality, such excitation possibly drive by the clinician's manipulation of the probe or tissues, the clinician's inflation or an inflatable balloon or membrane, the patient's manipulation in any manner of the probe or tissues, or the excitation of the probe or tissues using an onboard or mechanically coupled probe exciter means.

26) Any of the prostate probes herein wherein the patient's tissues are thermally manipulated by the probe or by an associated heating or cooling means, said thermal manipulation allowing for the improvement in a sought temperature or temperature related signal indicative of a tissue abnormality.

27) Any of the prostate probes herein wherein any collected or sensed data is registered to, overlaid upon or compared to a medical image of the prostate, that image or images taken at any time in any manner or taken in real time during the prostate probe exam.

28) Any of the prostate probes herein wherein any collected or sensed data is used to recommend the patient for follow-up examination using another diagnostic technique, modality, procedure or instrument.

29) Any of the prostate probes herein wherein a pressure/force sensor is backed by or mounted upon a selected hardness backer or substrate.

30) Any of the prostate probes herein wherein a temperature sensor is backed by a selected thermal conductivity backer or substrate or wherein two or more temperature sensing sub-elements are laterally thermally isolated.

31) Any of the prostate probes herein wherein any of:

(a) the probe is mounted to or worn upon a user's finger or fingers;

(b) the probe is worn by a user and covered during use with a sheath, condom, glove, isolation membrane, membrane, or balloon;

(c) a finger(s) worn or mounted probe includes a sensor backer of selected hardness or thermal conductivity; or

(d) a finger(s) worn or mounted probe includes any of: (i) a power line, (ii) a data connection, (iii) a fluid or gas lumen for any purpose, or (iv) a tissue heating or cooling means.

32) Any of the prostate probes herein wherein a temperature reading is sampled using an infrared window or window material which completely or substantially eliminates any air or gas gap in front of the tissue.

### Further Embodiments

We developed a further embodiment, which we believe to be novel over all prior art in all of its embodiments, that can be called a minimalist approach. This embodiment allows for both unaided-finger palpation and instrumented-finger palpation. In particular, this embodiment preferably utilizes a flex circuit based force or pressure sensor array that is capable of being located in two different positions relative to the examiner's finger. The first position permits instrumented-finger force or pressure mapping, whereas the second position permits unaided finger palpation or mapping.

We preferably utilize a long narrow flex sensor preferably capable of being tensionally physically pulled-back from the fingertip while under the glove. So in this preferred approach, the flex sensor is on the fingertip and the instrumented palpation is performed first. Then, preferably without removing the glove and possibly without removing the finger from the cavity, the flex sensor is pulled back from the fingertip along the practitioner's finger to expose the practitioner's bare (sensor-bare, not glove-bare) fingertip to the glove and anatomy as in the prior art. This thereby allows the examiner to conduct an unaided study immediately following his/her aided (flex-instrumented) study without intermediate delay. Within the scope of the invention is the reverse order wherein the bare-finger exam is done first, although it is somewhat more constraining to design a flex sensor that can be pushed out along the fingertip than one that can be pulled back from the fingertip.

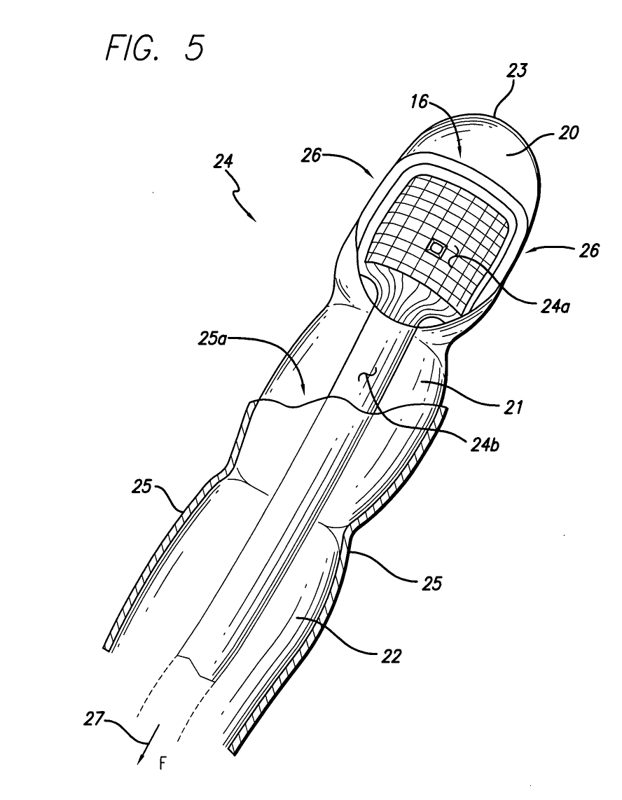

To assist in understanding this approach, we refer to FIG. 5, which is illustrates the flex-instrumented finger before rectal insertion. Shown is an examiner's finger with its three anatomical segments. Segment 20 is the fingertip portion and a bit of the fingernail 23 can be seen on the far side on its tip. Segment 21 is the midsegment and segment 22 is the segment attaching to the hand/palm. Thus, as should be clear, the articulating finger joints of interest for the invention are between segments 20/21 and 21/22.

The preferred force-sensor array flex circuit 24 is depicted with its sensor array portion 24a and its extended flex-trace section 24b. As an example, a preferred Tekscan sensor is model # 5800N having an array 24a of 100 force sensors arranged in a 10 by 10 square array roughly as depicted and having a maximum pressure range of 36 psi. Custom Tekscan sensors with somewhat lower pressure ranges (e.g., 5-15 psi) may provide even more sensitivity for light loading.

It will be noted that we have shown the overlying or covering surgical glove 25 fitted over the finger and flex circuit 24a/24b and that depicted glove 25 is shown broken away at edge 25a such that the exposed sensor region 24a can be seen in this Figure.

Moving now to the final major element, we depict an optional but preferred panel or sled part 26, which is shown underneath the flex sensor region 24a and on the fingertip tissue 20. We will discuss this sled component below in greater detail.

The flex force sensor lead or tail region 24b that routes the sensor electrical (or optical) traces to the outside world is preferably utilized to cause or drive the inventive sensor sledding-action or pullback action. A tensile force F (denoted 27) is applied such that the flex circuit parts 24a/24b slide backward away from the fingertip segment 20 to become resident in segment 21 (sensor not shown in withdrawn position) (or further back including to a totally removed state).

Means to pull on or otherwise tension the flex trace 24b segment are most easily made available where the flex segment exits the glove (not shown). Thus, one could pull on that exposed tensioning means as by the examiner using his second hand. In some cases, the present inventors expect that other means of pulling the flex back may be provided, including means that can be operated with the same (instrumented) hand or through the glove of the instrumented hand. A tensile or pulling action is preferred; however, pulling, pushing, rotating or twisting sensor region 24a in any direction is also within the inventive scope.

Thus, what we preferably have is a disposable flex/sled assembly 24a,24b/26. The purpose(s) of the sled element 26 are at least one or more of the following: a) to keep the sensor flex 24a from dislocating from the fingertip region 20 and desired orientation while it is needed for instrumented palpation, b) to keep the flex mechanically attached and preferably oriented to the finger while it is slid backwards (it preferably grips the body of the finger), and/or c) to provide a reproducible mechanical foundation for the flex force sensor's operation during the instrumented portion of the exam. After the instrumented palpation is completed, the flex sensor array 24a is pulled-back or sledded back such that the bare (preferably except glove) fingertip can then perform its uninstrumented exam. To do this, the examining hand/finger may or may not be removed from the rectum.

In this manner, the practitioner performs an instrumented recorded exam as well as his time-honored bare-finger gloved exam and can relate what he manually feels to what the instrumented exam told him.

Typically, the sled 26 and the flex 24a may be attached during the exam; however, they may be preattached as a fused, clamped or fastened pre-assembly or, alternatively, they may be fused, clamped or otherwise attached at exam time. Note that because of these choices, one may either have the sled 26 be disposable (with the flex-sensor, for example) or be reusable (with or without the flex-sensor).

The present inventors anticipate a kit having multiple size molded or formed sleds into which a flex is mounted at exam time and mechanically constrained (from at least backwards slippage relative to the sled 26). This may allow for flex/sled separation (disassembly), yet preserve the ability to pull back the assembly 24a/26 using the force F 27. One might also choose to provide different size sensors or have the sensors be physically trimmable for optimal patient fit.

Included in the inventive scope is the use of a glove 25 that beneficially restrains the flex/sled 24a/24b and 26 against the finger segments, yet still allows for the inventive sledding action toward the hand and away from the fingertip.

Although we have described a prostate exam, we emphasize that the inventive device may also be utilized for breast exams and testicular exams. In that case, one or more fingers may have one or more such devices. Further, in that case it may be the case, particularly for a self-exam, that a glove is not required. In any case, the inventors believe that for the breast application, it would be preferable to have a lubricant on the anatomy to enhance sliding of the instrumented fingertip(s) across the tissue to be examined.

We also include in our inventive scope a sled (actually a nonsliding clip) that does not slide but just clips to, grips or adheres to the finger. In this approach, one may clip on the sensor and use it then unclip it without necessarily sliding it along the finger. In this case, the “slider” can be made such that it does not easily slide on the finger and thus becomes a static clip. We have used the term “nonsliding clip” here simply to emphasize that the clip substantially replaces the slider.

In any event, during the instrumented exam, the sled (or clip) holds the sensor in the desired position and orientation, possibly with the help of an overlying glove or isolation membrane.

What is claimed is:

1. A prostate probe system for assessing one or both of BPH or prostate cancer comprising:
(a) a force or pressure sensor mounted on, in or to a rectally insertable probe and necessary means to power, connect to, switch and read data from the sensor;
(b) the force or pressure being measured during probe insertion from or at the rectal wall at two or more wall locations adjacent or juxtaposed to the prostate;
(c) the sensed force or pressure data from the two or more location's forming a force-map, pressure-map or data array relevant to the patient's underlying prostate condition or prognosis;
(d) the sensed data or force map being utilized at the time of the exam or later after the exam to compare the patients data to at least some other data from at least one other exam,
(e) a conclusion or recommendation regarding the patient's prostate condition thereby being providable by a practitioner to a patient based at least partly on a quantitative data comparison;
the probe optionally including one or more of:
(1) a mechanical exciter integrated in or capable of being mechanically coupled to the probe to excite at least a prostate anatomical portion;
(2) a motion, deflection, angle or inertial sensor of any type integrated in or couple able to the probe to track or monitor at least one probe or sensor position, orientation, angle, velocity or acceleration;
(3) a deflectable or inflatable balloon, membrane or mechanism capable of applying a load or deflection to one or more of the probe, the probe's sensor, or the anatomy to improve the probes performance or sensitivity; or
(4) an inflatable balloon or membrane used, at least in part, to measure a volume or compliance of any of a rectal wall or cavity or to heat or cool anatomy.

2. The probe system of claim 1 wherein any of the force or pressure readings are taken any of:
(a) while the patient and probe are essentially static or in mechanical equilibrium except for unavoidable perfusion and breathing motions;
(b) before, during or after the patient intentionally moves, distorts himself, squats, or bounces his anatomy or simulates a bowel movement;
(c) before, during or after the clinician moves or manipulates the probe or sensor manually, or with the aid of a probe-based sensor scanning means;
(d) before, during or after the clinician moves, manipulates, vibrates or oscillates the probe or anatomy with the optional deflectable or inflatable balloon, membrane or deforming mechanism;
(e) by moving the probe or its sensor in any translational, rotational or angular manner to take any of static, dynamic or transient readings;
(f) substantially simultaneously or in close temporal sequence from two or more different locations by two or more corresponding sensor sub-elements located, at the data-reading time, at those locations within a juxtaposed sensor array, some of those readings being at least one of static, dynamic or transient force or pressure readings;
(g) as triggered by a position or orientation sensor reading;
(h) as triggered by software;
(i) under a state of known prostate loading, excitation, oscillation or vibration; or
j) with accompanying patient identification data.

3. The probe system of claim 1 wherein the probe is or has at least one of:
(a) is finger(s) mounted or attached;
(b) is a handheld probe at least during insertion or removal;
(c) is a probe held by a patient-external exciter means;
(d) is a probe at least partially covered or wrapped in a condom, sheath or membrane while inserted;
(e) is a probe or has a probe sensor that is at least partially contained in, covered by, or manipulated by an inflatable membrane, balloon or deflecting or deforming mechanism;
(f) is a probe that can be immersed in at least one flowable gaseous or liquid-like inflating medium one or both of with or without a containment balloon or membrane;
(g) has a sensor array wrapped-upon, suctioned to, adhered to, fastened, clamped or clipped to or otherwise mounted to at least one surface portion or surface region of the probe;
(h) has a sensor array that is any of (i) 1×n sub-elements in length, (ii) m×n sub-elements in areal size, or (iii) includes a mechanically scannable sub-element or sub-element array;
(h) has a sensor which can operate in timed-coordination or synchrony with any of a) the operation of a mechanical exciter, b) the clinician's manipulation of the probe, c) the inflation or deflection of an inflatable balloon, membrane or mechanism, or d) software that scans or reads out the sensor; or
(i) includes a capacitive or resistive force or pressure sensor.

4. The probe system of claim 1 wherein the optional motion, deflection or inertial sensor is used for one of more of:
(a) to detect any operational parameter of an optional exciter;
(b) to control any operational parameter of an optional exciter;
(c) to detect or control a clinician's manipulation of the probe;
(d) to detect or control a patient's willful or unwillful movement of the probe or its adjacent anatomy;
(e) to verify or measure an exciter-induced vibration, oscillation or ring-down of a patient's anatomy;
(f) to achieve a desired rotation, angulation or translation rate of the probe or sensor; or
(g) to trigger the force/pressure sensor or sub-element(s) thereof to sample said forces or pressures.

5. The probe system of claim 1 wherein any of: (i) the probe system utilizes a spatial or spatial plus time coordinate system in its operation, (ii) the probe system utilizes a sensor or sensors having a sensor axis which is aligned to a probe body axis, or (iii) a sensor is wrapped around or upon any portion of the probe.

6. The probe system of claim 1 wherein any of:
(a) a sensor or sensor array is fabricated utilizing flex circuit or lithographic technologies;
(b) a sensor or sensor array utilizes capacitive or resistive sub-element(s);
(c) a sensor or sensor array is read out, fully or in part, at a controlled sub-element read rate or sensor array frame rate
(d) a sensor or sensor array has electrically or optically addressable sensor sub-elements for reading purposes;
(e) a sensor or sensor array is disposable after a recommended number of uses of one or greater or after a system-enforced number of uses;
(f) a sensor or sensor array is larger or longer in at least one dimension than an anticipated tumor that might be detected;
(g) a sensor or sensor array is larger in at least one dimension than a prostate gland dimension which can be sensed at the rectal wall;
(h) any portion of a sensor or sensor array element or sub-element(s) is read as triggered or gated by a clock or by read data from the optional motion, deflection or inertial sensor;
(i) maximum or minimum static, dynamic or transient force or pressure readings are any of detected, recorded or compared; or
j) any of the force/pressure absolute values or derivatives or slopes of such data or data-graphs are utilized in determining an extent of prostate enlargement or tumor-presence likelihood.

7. The probe system of claim 1 wherein a force or pressure sensing element, sub-element or array of such sub-elements is situated, held, suctioned, adhered, clipped, clamped or mounted upon a foundation or backing material having stiffness or rigidity larger than that of the typical tissues being examined, the tissue force variation thereby being substantially preserved for detection by avoiding conformational relaxation of the sensor shape itself.

8. The probe system of claim 1 wherein a sensor element, sub-element or sub-element array has at least one curved dimension or plane of curvature which enhances the probes force/pressure measurement or measurement-range performance.

9. The probe system of claim 8 wherein said at least one plane of curvature at least one of:
(a) generally conforms to a typical healthy anatomy;
(b) fits or conforms to a healthy anatomy in a manner presenting a substantially uniform or a substantially normal healthy force/pressure map;
(c) allows for a smooth or comfortable probe insertion or manipulation.
(d) assures that despite the tissues of interest being of irregular shape that all such areas contact the sensor and provide a useful pressure/force reading; or
(e) causes indentation palpation of the prostate in a manner similar to that of a pressing fingertip.

10. The probe system of claim 1 wherein a force/pressure sensor array has at least one radius or curvature in one plane which is substantially larger or more gentle than that of the first inserted probe body radius or finger radius.

11. The force/pressure probe system of claim 1 wherein:
(a) some static force/pressure readings are taken;
(b) some dynamic or transient readings are taken;
(c) both some static and dynamic or transient readings are taken;
(d) some maximum or minimum readings are taken or are reported;
(e) one or more sensing elements or sub-elements takes readings at two or more times;
(f) two or more sensing elements or sub-elements at different locations are read at the same or different times; or
(g) which element or sub-element(s) is/are read is determined, at least in part, by a probe orientation, a known load on the probe, a state of mechanical excitation of the probe and/or the anatomy or a software program.

12. The probe system of claim 1 wherein any of:
(a) the probe is capable of being powered by an internal energy storage means;
(b) the probe is capable of being powered by an external energy storage or source means;
(c) the probe is capable of being used with a rechargeable or reenergizable energy storage means; or
(d) the probe has any of a wired, wireless or lumen fluid/gas connection to any of a support utility, console or to a network.

13. The probe system of claim 1 wherein at least one measured, sensed, detected or saved force or pressure reading at one or more sensor elements or sub-elements is at least one of:
(a) a substantially static force or pressure;
(b) a substantially dynamic force or pressure sensed during a mechanical loading or excitation of the probe or of the adjacent anatomy;
(c) a substantially transient force or pressure sensed after a removal, variation in or change in a static or dynamic mechanical loading or excitation;
(d) a force or pressure on an upwards or increasing amplitude slope;
(e) a force or pressure on a downwards or decreasing amplitude slope;
(f) a force or pressure having a known time-phase relationship with a static, dynamic or transient loading or excitation;
(g) a force or pressure nearing or at a peak or minimum value;
(h) a spatially or time-averaged force or pressure from one or more sensor elements or sub-elements, said elements not necessarily being adjacent ones;
(i) a force or pressure determined to be outside of or inside of a range; or
(j) a force or pressure which has substantially settled to a constant value after a transient or waiting period.

14. The probe system of claim 1 wherein a patient is examined at two or more points in time, including wherein said two or more points comprise two or more sequential scheduled exams or two or more exams on the same day, said results from at least some of the exams being compared to each other or to a those of a larger population.

15. The probe system of claim 1 wherein the exam is performed by any of a doctor, clinician, technician or patient.

16. The probe system of claim 1 wherein the patient receives a medicament or drug which enhances the sought signal of a prostate tissue abnormality or which calms or soothes the patient.

17. The probe system of claim 1 wherein the patient has his tissues manipulated or excited in a mechanical way to enhance the sought signal of a prostate tissue abnormality, said excitation possibly driven by the clinician's manipulation of the probe or tissues, the patient's manipulation of the probe or tissues, or the excitation of the probe or tissues using a probe exciter means.

18. The probe system of claim 1 wherein the patient's tissues are thermally manipulated by the probe or by an associated heating or cooling means, said thermal manipulation allowing for the improvement in a sought force/pressure signal indicative of a tissue abnormality.

19. The probe system of claim 1 wherein some collected or sensed data is registered to, overlaid upon or otherwise compared or correlated to a medical image of the prostate, that image or images taken at any time in any manner or taken in real time during the probe system exam.

20. The probe system of claim 1 wherein some collected or sensed data is used to recommend the patient for follow-up examination using another diagnostic technique, modality, procedure or instrument, said recommendation being deliverable to the patient either at exam time or some amount of time after the exam.

21. The probe system of claim 1 wherein the probe also measures ringdown of vibrating tissues or tumors in any manner, said ringdown time(s) providing an indication of prostate health.

22. The probe system of claim 1 wherein any one or more of:
(a) a removable sheath, membrane, condom or bladder is utilized;
(b) any inflatable entity is utilized for a probe/sensor loading or probe/sensor fixation purpose;
(c) any inflatable entity or cavity containing a flowed material is utilized to control or manipulate an anatomical temperature;
(d) a deformable or inflatable member is utilized which applies a load on a rectal wall or on a prostate organ;
(e) a force or pressure sensor is disposed or abutted to or on a foundation or backer which is at least twice as hard as tissue and more preferably at least ten times as hard as tissue;
(f) a force or pressure sensor is used which has a dimension larger than a tumor dimension or larger than a prostate dimension;
(g) a force or pressure sensor is used which is mounted on or to a flat, curved or convex surface, said flatness, curvature or convexity being at least in one plane;
(h) a force or pressure sensor is used which has a sub-sensor or pixel pitch of less than or equal to 3 mm and more preferably less than or equal to 2 mm; or
(i) a force or pressure sensor is used which is mounted on a temperature controlled foundation or probe.

23. A probe system for assessing one or both of BPH or prostate cancer coming:
(a) a temperature sensor mounted on, in or to a rectally insertable probe and necessary means to power, connect to, switch and read data from the sensor;
(b) the temperature sensor being employed to sample two or more rectal wall locations adjacent or juxtaposed to the prostate and from the rectal wall during probe insertion or while the probe is inserted;
(c) the sensed data from the two or more location's forming a temperature-map or temperature data-array having relationship to the patient's underlying prostate;
(d) the sensed temperature data being utilized at the time of the exam or later after the exam to compare the patients data to at-least some other data from at least one other exam; and
(e) a conclusion or recommendation regarding the patients prostate condition thereby being providable by a practitioner to a patient based at-least partly on a quantitative data comparison;
probe optionally including one or more of:
(1) a motion, deflection, angle or inertial sensor of any type integrated in or coupleable to the probe to track or monitor at least one probe or sensor position, orientation, angle, velocity or acceleration;
(2) a deflectable or inflatable balloon, membrane or mechanism capable of applying a load or deflection to one or more of the probe, the probe sensor, or the anatomy to improve the probes performance or sensitivity; or
(3) a means of injecting or removing heat from a tissue region of interest.

24. The probe system of claim 23 wherein any one or more of:
(a) a temperature measurement or sensing event utilizes a thermally contacting sensing means including any of a thermocouple, thermistor, diode or precision resistor;
(b) a temperature measurement or sensing event utilizes any type of optical sensing means including mid or near infrared optical means;
(c) an optical temperature measurement or sensing means utilizes one or more of: a gaseous standoff gap; an optically transparent window standoff material of any solid or liquid-like type, whether the window material contacts the tissue itself or not;
(d) a two or three dimensional array of temperature detection sub-elements is provided; or
(e) one or more temperature detection elements or sub-elements utilizes an optical component to achieve spatial scanning.

25. The probe system of claim 23 wherein any one or more of:
(a) two temperatures taken at two different times are recorded, compared or reported;
(b) two temperatures taken at two different tissue locations are recorded, compared or recorded;
(c) a maximum or minimum temperature at a tissue location is recorded, compared or reported;
(d) an increasing or decreasing temperature at a tissue location is recorded, compared or reported;
(e) the slope of a temperature change at least one tissue-location or region of locations is computed, recorded, compared or reported; or
(f) a substantially static, dynamic or transient temperature or temperature change-rate is computed, recorded, compared or reported.

26. The probe system of claim 23 wherein any of:
(a) substantially rectal wall surface temperatures are detected or measured;
(b) substantially underlying subsurface temperatures are detected or measured; or
(c) a tissue temperature can be manipulated favorably using a probe system or probe-related heated or cooling means, favorably meaning allowing for a better signal-to-noise ratio of the temperature measurement signal being sought.

27. The probe system of claim 23 wherein any of the listed optional features allows for any one or more of (a) spatial motion control of the probe, (b) improved temperature accuracy or improved spatial accuracy of temperature patterns sampled from the anatomy, (c) determination or control of temperature sampling sites or locations, or d) triggering of temperature data taking at least one sensor sub-element.

28. The probe system of claim 23 wherein the probe is one of (a) a finger(s)-mounted or attached probe, or (b) a standalone probe which is itself insertable in the anatomy, (c) an at least in part disposable probe, (d) a probe that is protected during use by a sheath, membrane or condom which is arranged not to substantially interfere with temperature mapping, (e) a practitioner manipulatable probe, or (f) a probe that any of records or transmits data in a wired or wireless fashion.

29. The probe system of claim 23 wherein a patient is examined at two or more points in time, including wherein said two points comprise two sequential scheduled exams, said results from the exams being compared to each other or to a those of a larger population, said two or more sequential exams being on the same day or different days.

30. The probe system of claim 23 wherein the exam is performed by any of a doctor, clinician, technician or patient.

31. The probe system of claim 23 wherein the patient receives a medicament or drug which enhances the sought signal of a prostate tissue abnormality or which calms or soothes the patient.

32. The probe system of claim 23 wherein the patient has his tissues manipulated or excited in a mechanical way to enhance the sought signal of a prostate tissue abnormality, said excitation possibly drive by the clinician's manipulation of the probe or tissues, the patient's manipulation of the probe or tissues, or the excitation of the probe or tissues using the probe exciter means.

33. The probe system of claim 23 wherein the patient's tissues are thermally manipulated by the probe or by an associated heating or cooling means, said thermal manipulation allowing for the improvement in a sought temperature signal indicative of a tissue abnormality.

34. The probe system of claim 23 wherein some collected or sensed data is registered to, overlaid upon or correlated-to a medical image of the prostate, that image or images taken at any time in any manner or taken in real time during the temperature probe exam.

35. The probe system of claim 23 wherein some collected or sensed data is used to recommend the patient for follow-up examination using another diagnostic technique, modality, procedure or instrument.

36. The probe system of claim 23 wherein the probe also measures ringdown of tissues or tumors therein in any manner.

37. The probe system of claim 23 wherein any one or more of:
(a) a removable sheath, membrane, condom or bladder is utilized;
(b) any inflatable entity is utilized for a loading or fixation purpose;
(c) any inflatable entity of cavity containing a flowed material is utilized to control a temperature;
(d) a deformable or inflatable member is utilized which applies a load on a rectal wall or on a prostate organ;
(e) a force or pressure sensor is disposed or abutted to a foundation which is at least twice as hard as tissue and more preferably at least ten times as hard as tissue;
(f) a force or pressure sensor is used which has a dimension larger than a tumor dimension or larger than a prostate dimension;
(g) a force or pressure sensor is used which is mounted on or to a flat, curved or convex surface, said flatness, curvature or convexity being at least in one direction;
(h) a force or pressure sensor is used which has a sub-sensor or pixel pitch of less than or equal to 3 mm and more preferably less than or equal to 2 mm;
(i) a force or pressure sensor is used which a contacting temperature sensor is mounted on a temperature controlled foundation or probe; or
(j) a temperature sensor utilizes infrared optical light.

38. A combined prostate probe system having both force/pressure detection capability and temperature detection capability for assessing one or both of BPH or prostate cancer comprising:
(a) a force or pressure sensor mounted on, in or to a rectally insertable probe and necessary means to power, connect to, switch and read the sensor;
(b) the force or pressure being employed to sample two or more rectal first wall locations adjacent or juxtaposed to the prostate during probe insertion or while the probe is inserted;
(c) a temperature sensor mounted on, in or to a rectally insertable probe and necessary means to power, connect to, switch and read the sensor;
(d) the temperature sensor being employed to sample two or more second rectal wall locations adjacent or juxtaposed to the prostate during probe insertion or while the probe inserted;
(e) the sensed data from the first locations and the second locations being utilized to form a force-map, pressure-map or force/pressure data array as well as a temperature-map or temperature data-array each having relationship to the patient's underlying prostate, the first and second locations being the same or different locations;
(f) the quantitative force/pressure and temperature data being compared or correlated with similar data from at-least one other exam or exam database; and
(g) the comparison or correlation being utilized to judge a state of prostate health or condition;
the probe optionally including one or more of:
(1) a mechanical exciter integrated in or capable of being coupled to the probe;
(2) a motion, deflection, angle or inertial sensor integrated in or couple able to the probe;
(3) a deflectable or inflatable balloon, membrane or mechanism capable of applying a load or deflection to one or both of the probe or the anatomy; or
(4) a means of injecting or removing heat from a tissue region of interest.

39. The combined prostate probe system of claim 38 wherein said probe also allows for at least one of:
(a) physical registration of the force/pressure data and the temperature data if both data types are taken;
(b) sampling or detection of force/pressure data and temperature data from a combined or interdigitated sensor or sensor(s);
(c) taking of either or both data types in any desired sequential, parallel or time-interleaved sequence;
(d) the ability to detect an undesirable tissue condition evidenced both by a pressure/force anomaly and a temperature anomaly;
(e) immediate or follow-up reporting to a patient as to how he compares to a database of prior exams done on one or more persons; or
(f) data network connectivity for any reason.

40. The combined prostate probe system of claim 38 wherein said probe is one or more of:
(a) finger(s) mounted or attached;
(b) a standalone probe itself capable of being inserted;
(c) has one or more exchangeable sensors including at least one force/pressure sensor and/or one temperature sensor;
(d) has one or more disposable sensors or sensor types;
(e) utilizes a reusable sensor or handle;
(f) during measurement is covered by a sheath, condom or membrane which is arranged not to substantially interfere with said measurement(s);
(g) has a force/pressure sensor in one probe region and a temperature sensor in a second probe region, the two regions preferably being opposed regions presentable to target tissues via rotation or angulation of the probe;
(h) is wipeable or immersable in a liquid, gaseous or plasma sterilant or antiseptic without a covering condom, membrane or sheath installed;
(i) contains or is connectable to a power source; or
(j) contains or is connected to a wired or wireless data network or a data recording means.

41. The combined prostate probe system of claim 38 wherein a patient is examined at two or more points in time, including wherein said two points comprise two sequential scheduled exams, said results from the exams being compared to each other or to a those of a larger population, said sequential exams being on different days or on the same day.

42. The combined prostate probe system of claim 38 wherein the exam is performed by any of a doctor, clinician, technician or patient.

43. The combined prostate probe system of claim 38 wherein the patient receives a medicament or drug which enhances the sought signal of a prostate tissue abnormality or which soothes or calms the patient.

44. The combined prostate probe system of claim 38 wherein the patient has his tissues manipulated or excited in a mechanical way to enhance the sought signal of a prostate tissue abnormality, said excitation possibly drive by the clinician's manipulation of the probe or tissues, the patient's manipulation of the probe or tissues, or the excitation of the probe or tissues using any probe mechanical exciter means.

45. The combined prostate probe system of claim 38 wherein the patient's tissues are thermally manipulated by the probe or by an associated heating or cooling means, said thermal manipulation allowing for the improvement in a sought signal indicative of a tissue abnormality.

46. The combined prostate probe system of claim 38 wherein some collected or sensed data is registered to, overlaid upon or correlated to a medical image of the prostate, that image or images taken at any time in any manner or taken in real time during the probe exam.

47. The combined prostate probe system of claim 38 wherein some collected or sensed data is used to recommend the patient for follow-up examination using another diagnostic technique, modality, procedure or instrument.

48. The combined prostate probe system of claim 38 wherein the probe also measures ringdown of mechanically excited prostate-relevant tissues or tumors in any manner.

49. The combined prostate probe system of claim 38 wherein any one or more of:
(a) a removable sheath, membrane, condom or bladder is utilized;
(b) any inflatable entity is utilized for a loading or fixation purpose;
(c) any inflatable entity or cavity containing a flowed material is utilized to control a temperature;
(d) a deformable or inflatable member is utilized which applies a load on a rectal wall or on a prostate organ;
(e) a force or pressure sensor is disposed or abutted to a foundation which is at least twice as hard as tissue and more preferably at least ten times as hard as tissue;
(f) a force or pressure sensor is used which has a dimension larger than a tumor dimension or larger than a prostate dimension;
(g) a force or pressure sensor is used which is mounted on or to a flat, curved or convex surface, said flatness, curvature or convexity being at least in one plane;
(h) a force or pressure sensor is used which has a sub-sensor or pixel pitch of less than or equal to 3 mm and more preferably less than or equal to 2 mm;
(i) a force or pressure sensor is used which a contacting temperature sensor is mounted on a temperature controlled foundation or probe; or
(j) a temperature sensor utilizes infrared optical light.

50. A probe system for evaluating a condition of a prostate gland including:
(a) a rectally insertable probe;
(b) a means to excite the tissue of interest into an excited motion state; and
(c) a means to stop said excitation and monitor the decaying ringdown or attenuation of the vibrating tissue portions, the ringdown behavior giving information related to the health of the prostate such as a state of enlargement or presence of tumors.

51. The probe system of claim 50 wherein any of:
(a) the excitation is provided by the patient's motion or simulated bowel movement;
(b) the excitation is provided by a clinician's manipulation of tissues or of the probe;
(c) the exciter is integrated into, attached to or physically coupled to the probe;
(d) the exciter can excite the tissues using at least one of an impulse or at least one multiwave cyclic frequency;
(e) the ringdown is monitored by at least one of a force/pressure sensor or by an integrated or coupled acceleration, displacement, angle or vibration sensor;
(f) the ringdown is monitored by a MEMs sensor;
(g) the ringdown is evaluated at two or more excitation frequencies; or
(h) the ringdown is evaluated using a broadband excitation impulse.

52. The probe system of claim 50 wherein the probe also measures ringdown of tissues or tumors therein in any manner.

53. The probe system of claim 50 wherein any one or more of:
(a) a removable sheath, membrane, condom or bladder is utilized;
(b) any inflatable entity is utilized for a loading or fixation purpose;
(c) any inflatable entity of cavity containing a flowed material is utilized to control a temperature;
(d) a deformable or inflatable member is utilized which applies a load on a rectal wall or on a prostate organ;
(e) a force or pressure sensor is disposed on or abutted to a foundation or mechanical backer that is at least twice as hard as a measured tissue;
(f) a force or pressure sensor is used which has a dimension larger than a tumor dimension or larger than a prostate dimension;
(g) a force or pressure sensor is used which is mounted on or to a flat, curved or convex surface, said flatness, curvature or convexity being at least in one plane;
(h) a force or pressure sensor is used which has a sub-sensor or pixel pitch of less than or equal to 3 mm and more preferably less than or equal to 2 mm;
(i) a force or pressure sensor is used which a contacting temperature sensor is mounted on a temperature controlled foundation or probe; or
(j) a temperature sensor utilizes infrared optical light.

54. A force or hardness mapping device for palpation-examination of patient anatomical tissues for abnormalities or assessing states of firmness comprising:
(a) a force or pressure sensor having at least one sensing element;
(b) the sensor capable of palpating a patient's anatomy while at an examiner's fingertip palpation position and situated under an examiner's glove;
(c) the sensor capable of being parked or removed to a position away from the examiner's palpating fingertip such that the examiner's bare fingertip is also operable to palpate the patient's anatomy also through the glove; and
(d) the sensor being movable between a fingertip position and a removed position at least once in one direction.

55. The device of claim 54 wherein any one or more of:
(a) the fingertip sensor position is first used to perform the sensor-supported exam-with or without an overlying glove or sheath;
(b) the removed or parked position is used second in order to allow for the sensor-free fingertip to perform a bare-finger palpation-with or without an overlying glove or sheath;
(c) the sensor is slid along a length dimension of the finger between two such positions;
(d) the sensor is pulled along a length dimension of the finger between two such positions;
(e) the sensor is pushed along a length dimension of the finger between two such positions;
(f) the sensor is twisted or rotated in any direction to enable a sensor-free fingertip palpation;
(g) the sensor has two or more force or pressure sensor sub-elements arranged in a pattern;
(h) the sensor has two or more force or pressure sensor sub-elements arranged in an array;
(i) the sensor has a sensor element(s) region and a connecting trace-routing region;
(j) a sled is provided which grips, clamps, is-fastened to or is adhered to at least one of the sensor or then examiners finger;
(k) a sled is provided which any of grips, clamps, is-fastened to or is adhered to both the sensor and the examiners finger-the attachment means possibly being different for each;
(l) the sled slides or rotates along or across at least one examiners finger portion;
(m) the sled slides or rotates along or across a glove surface-preferably an interior glove surface;
(n) the sled and the sensor are a prejoined subassembly;
(o) the sled and the sensor are joined at exam time;
(p) one or more sleds and one or more sensors forms a kit-optionally also containing one or more gloves;
(q) a sliding or rotating interface between finger/sensor or sensor/glove or finger/glove is lubricated in any manner;
(r) at least one of the sled, sensor or glove is disposable;
(s) the sled and sensor are disposable-regardless of their state of temporary or permanent attachment;
(t) the examiner pulls the sensor back away from the examining fingertip using any of i) the thumb of his examining hand, ii) any portion of his other hand;
(u) the sensor utilizes flexible circuit, MEMS or lithographic technologies in its fabrication; or
(v) a breast, prostate or testicle is being examined.

56. The device of claim 54 wherein any one or more of:
(a) multiple sleds or size/shapes/choices of sleds are provided in a kit;
(b) a reusable sled is employed;
(c) a sled elastically or spring-wise grips a finger portion;
(d) a sled has a controlled hardness or flexural rigidity;
(e) a sled has a hardness or flexural rigidity which: i) substantially rigidizes the sensor for at least a period or ii) it partially rigidizes the sensor for at least a period;
(f) a sled is molded, shaped, formed or fitted at any stage of manufacture, preparation or use;
(g) a sled is fabricated, at least in part, of a polymeric, metallic or ceramic material;
(h) a sled has variable rigidity and said rigidity is controllably variable by the examiner;
(i) the sled is permanently fastened to the sensor during manufacture; or
(j) the sensor is mechanically mounted into the sled before use with the possible help of sled mechanical features including slots, channels, clips, clamps, fasteners, springs and elastomeric members.

57. The device of claim 54 wherein any one or more of:
(a) during the instrumented sensor-based palpation the sensor output is any of annunciated, displayed, recorded or passes over a wired or wireless (including optical) network;
(b) during the uninstrumented sensor-free palpation the examiner compares or has compared for him/her his manual palpation results with at least some instrumented sensor results;
(c) the examiner utilizes a 3D positioning system such that the sensor location is known such that positional information can be employed for making anatomical hardness maps or for comparing said maps;
(d) the results of a prior exam are compared to the results of a current exam;
(e) the results from a patient are compared to the results of a population of patients; or
(f) a single gloved exam allows for instrumented and uninstrumented palpation.

58. The device of claim 54 wherein the sled is any one or more of:
(a) wrappable around an examiners finger segment in any manner such that it is retained substantially in place while that is desired;
(b) receptive of an inserted sensor such that said sensor can be substantially retained in place relative to the sled;
(c) capable of gripping the examiners finger in a manner such that it can both retain the fingertip sensor-mapping position yet still be moved to a removed position without becoming decoupled from said finger during said moving; or
(d) one or both of the sensor and sled wherein they have a conformal shape to the finger and don't easily hang-up on the glove or on the patients tissues.

59. The device of claim 54 wherein the sensor works on variable capacitance, variable resistance, variable-conductance or optical-parameter variation mechanisms.

60. The device of claim 54 wherein the examiner also utilizes an ultrasound imaging device mounted on his fingertip.

61. The device of claim 54 wherein a patient's prostate, breast, or organ is examined.

62. The device of claim 54 wherein a glove or finger sheath is provided separately from the device or is provided with the device.

63. The device of claim 54 wherein a glove or finger sheath is provided to the examiner already mated to one or both of the sensor or to any sled which may be used.

64. The device of claim 54 wherein the sled and the glove are a prefabricated subassembly.

65. The device of claim 54 wherein any part of a glove, sensor or any sled used has a lubricant, adhesive or gripping surface to either cause sliding or to prevent sliding of a first surface relative to a second surface.

66. The device of claim 54 wherein a glove or sheath is fitted over the instrumented finger while said glove or sheath is stretched and is then allowed to collapse in tension upon the finger/sensor.

67. The device of claim 54 wherein said sensor movability includes at least one translation or rotation of the sensor relative to the fingertip.

68. The device of claim 54 wherein said sensor is moved by application of a pulling or tensile force, a pushing force or a twisting or torque applied, directly or indirectly, by the examiner.

69. The device of claim 54 wherein said force or torque is applied to at least one of a sensor or a sled.

70. The device of claim 54 wherein said force or torque is mechanically communicated through at least one of:
(a) a sensor flex circuit portion;
(b) a sensor flex circuit trace region;
(c) an activation wire, string, chain or cable;
(d) a hydraulic or pneumatic means including a vacuum means; or
(e) an elongated mechanical element fitted along or passing along a finger length dimension-such a flexible or semirigid rod or bar.

71. The device of claim 54 wherein said motion is in one direction only.

72. The device of claim 54 wherein said motion is in either direction.

73. The device of claim 54 wherein said motion is in both directions.

74. The device of claim 54 wherein the sensor presents an array of sensing sub-elements to the anatomy being studied.

75. The device of claim 54 wherein the examiner one or both of presses the sensor against the anatomy being studied or slides the sensor across anatomy being studied.

76. The device of claim 54 wherein a force, pressure or hardness map is displayed or recorded-said map being at least a map of tissue in contact with the sensor at a moment in time-said sensor possibly being smaller in lateral dimension than the overall lateral dimension or extent of the anatomy to be examined.

77. The device of claim 76 wherein the sensor is smaller than the anatomy to be examined thereby requiring movement of the sensor across the anatomy to capture mapping data of the whole anatomical target.

78. The device of claim 76 wherein the sensor is about the same size or is bigger than the anatomical target such that large lateral sliding or movement of the sensor is not required to capture the force map from the entire anatomical region of interest.

79. The device of claim 78 wherein a console or system paints an extended map of the entire anatomy based on data gathered from two or more sensor locations or orientations.

80. The device of claim 79 wherein the console or system deduces sensor position from the pattern of force data passing across its sensing surface.

81. The device of claim 79 wherein the console or system deduces sensor position utilizing a spatial positioning system such as a magnetic, electromagnetic or acoustic positioning system.

82. The device of claim 54 supplied in kit form and including a disposal bag or container for the used device.

83. The device of claim 54 including or coupled to a use-limiter which prevents multiple and unsafe reuse of the device.

84. The device of claim 54 wherein a wireless connection is provided between the device and a remote receiver or console, the wireless capability eliminating the need for a long connecting cable or umbilical connected to the device.

85. The device of claim 54 provided for home use.

86. The device of claim 54 where at least one temperature sensor is also provided, with at least one temperature reading from said at least one temperature sensor providing additional data as to the health condition of the prostate.

87. The device of claim 85 wherein the device is capable of, directly or indirectly, making its mapped data available for transmission over a network such as the internet.

88. The device of claim 85 wherein a patient follows remotely provided or software-delivered instructions for use, said instructions optionally being adaptive to results being measured or not measured.

89. A force or hardness mapping device for palpation-examination of patient anatomical tissues for abnormalities or for assessing states of firmness comprising:
(a) a force or pressure sensor having at least one sensing element;
(b) the sensor capable of palpating a patient's anatomy while mounted to or on an examiner's palpating fingertip, while situated under an examiner's glove;
(c) the sensor having connecting traces routed down the examiner's finger away from the palpating fingertip; and
(d) the sensor capable of being clipped, clamped or fastened to the examiner's palpating fingertip, optionally with a backer material or component at least temporarily.

90. The device of claim 89 wherein the sensor is moved under the examiner's glove between at least two positions, one position allowing for instrumented palpation, and the other position allowing for uninstrumented palpation.
